(19) United States
(12) Patent Application Publication
(10) Pub. No.: US 2024/0295495 A1
(43) Pub. Date: Sep. 5, 2024

(54) SYSTEMS FOR FACILE VIRAL CLEARANCE VALIDATION THROUGH THE DEVELOPMENT OF FLUORESCENT VIRAL SURROGATES

(71) Applicant: Rensselaer Polytechnic Institute, Troy, NY (US)

(72) Inventors: Todd Michael PRZYBYCIEN, Milton, VT (US); Runye Helen ZHA, Troy, NY (US); Hannah Rose DOSS, Troy, NY (US)

(73) Assignee: Rensselaer Polytechnic Institute, Troy, NY (US)

(21) Appl. No.: 18/388,130

(22) Filed: Nov. 8, 2023

Related U.S. Application Data
(60) Provisional application No. 63/547,563, filed on Nov. 7, 2023.
(60) Provisional application No. 63/423,568, filed on Nov. 8, 2022.

(51) Int. Cl. G01N 21/64; C12Q 1/06; G01N 33/15

(52) U.S. Cl. CPC G01N 21/6428; C12Q 1/06; G01N 33/15; G01N 2333/005; G01N 2500/20

(57) ABSTRACT

Evaluating viral clearance of a sample including a drug of interest is performed via modified viral surrogate nanoparticles that mimic a target live virus equivalent. The nanoparticles include fluorescent materials and a viral surface-mimicking layer that physicochemically mimics the external surface of the target live virus equivalent. One or more capsid proteins of the live virus are bound to the nanoparticle core (for non-enveloped viruses) or incorporated into a lipid bilayer (for enveloped viruses). A process solution is formed by adding the nanoparticles to the sample. The solution is subjected to purification steps to eliminate impurities, forming a product process solution. The product process solution is filtered through a dead-end flow nanofiltration membrane separator configured to bind the fluorescent nanoparticles. A load process solution is filtered as well. Baseline decomposition of the fluorescence intensity measurements from the separate membranes can, upon application of a standard curve indicate the relative nanoparticle concentration and thus the efficacy of the purification steps against the target live virus equivalent.

### CROSS REFERENCE TO RELATED APPLICATION(S)

This application claims the benefit of U.S. Provisional Application Nos. 63/423,568, filed Nov. 8, 2022, and 63/547,563, filed Nov. 7, 2023, which are incorporated by reference as if disclosed herein in their entireties.

### BACKGROUND

For biologic drugs manufactured via mammalian cell lines, endogenous or adventitious viral contamination is a threat and can have serious consequences for patient safety, drug supply, and production. To prevent contamination, regulatory agencies require the execution of viral clearance studies to assure that downstream purification processes can clear several orders of magnitude of virus, should an undetected contamination event occur during cell culture. Viral clearance validation is currently completed by performing live virus spiking studies, where high concentrations of live virus are introduced into drug-containing load materials and clearance capacity is evaluated across each downstream unit operation. Alternatives to using live virus are highly desirable, as live viral spiking studies are typically performed off-site, taking months to perform, costing hundreds of thousands of dollars, and often posing high risk of error with respect to preservation of virus-containing samples and quantification of viral content. Recent attempts have been made to develop model surrogate virus particles for use in viral clearance validation; these surrogates however either lack the ability to specifically mimic live model viruses, or employ cumbersome quantification strategies with narrow ranges of sensitivity not representative of live virus quantification.

The most representative way of evaluating viral clearance in current downstream processes is by performing live virus spiking studies. Viral spiking studies involve introducing a known amount or concentration of representative, live virus into the product-containing load material of the downstream steps of interest. The unit operation is executed, and the amount or concentration of persisting virus in the product stream is determined. A virus logarithmic reduction value is then calculated as the difference in the logarithm of the infectious viral particle amount or concentration in the load minus that in the product-containing efflux. Virus concentration is measured via either plaque titer or TCID₅₀ assays and involves culturing indicator cell lines with the virus-containing process samples for 14-28 days to allow for virus propagation, cytopathic effect development, and plaque development. Plaque titer assays determine the concentration of lytic viruses through quantification of plaques (dead cells) in a monolayer culture resulting from the application of a sample of a known volume and dilution, where 1 plaque=1 infectious viral particle per sample. TCID50 assays determine the sample dilution required to produce observable morphological changes, assessed via optical microscopy, in 50% of the indicator cells. When working with the model live viruses used in spiking studies, a Biosafety Level (BSL) 2 environment is required. Because BSL-2 laboratories are less common in industrial process research and development settings than BSL-1, and also because said labs are often located at the same facility as the manufacturing plant, live virus spiking studies cannot be run in-house. See, e.g., Food and Drug Administration. Guidance for Industry Q5A Viral Safety Evaluation Derived From Cell Lines of Human or Animal Origin Guidance for Industry; 1998; World Health Organization. Guidelines on viral inactivation and removal procedures intended to assure the viral safety of human blood plasma products; World Health Organization. Recommendations for the Evaluation of Animal Cell Cultures as Substrates for the Manufacture of Biological Medicinal Products and for the Characterization of Cell Banks. 2010, No. October, etc. This results in highly expensive collaborations, i.e., hundreds of thousands of dollars with contract research organizations (CROs) to complete this work.

As prescribed by regulatory agencies, viral spiking studies are required as Investigational New Drug (IND) and Biologics Licensing Application (BLA) filing components. For the IND, simpler two-model virus studies using new purification media, i.e., membranes or chromatography resin, are performed. Pre-BLA studies are more complex, employing four model viruses using both new and aged purification media to evaluate any changes in viral clearance capability over the media lifetime. Regulatory agencies do not require spiking studies for every downstream unit operation. Rather, spiking studies are typically performed on steps expected to provide at least 1 log PFU/mL clearance, i.e., a log reduction value of at least one. Viral clearance across viral inactivation (VI) and viral filtration (VF) processes, denoted as the “viral clearance dedicated steps”, are often evaluated in a separate study.

Regulatory agencies recommend that model viruses are selected that represent endogenous and adventitious viruses, DNA and RNA viruses, and enveloped and non-enveloped viruses. Endogenous viruses are made by the host cell through evolutionary inclusion of viral DNA in the host's natural genome; these viruses are typically retrovirus-like particles. Adventitious viruses are introduced into the production process as a contaminant, typically during the upstream process via contaminated raw materials or exposure of materials and equipment to workers shedding virus. Recently, data compiled from regulatory viral clearance databases showed that the most common model viruses employed for IND submission are retrovirus (RNA, enveloped) and parvovirus (DNA, non-enveloped), and for BLA submission are retrovirus, parvovirus, herpesvirus (DNA, enveloped), and reovirus (RNA, non-enveloped). These model viruses are chosen based on the extent to which retrovirus can mimic endogenous retrovirus-like particles, and how parvovirus, herpesvirus, and reovirus can serve as general model adventitious viruses. Parvoviruses in particular are also chosen due to their small size and the challenge they pose to removal via viral filtration. When working with CHO cell lines, minute mouse virus (MVM) is often chosen as the model parvovirus, as MVM is a frequent contaminant in rodent cell lines due to rodent fecal contamination of raw materials.

Non-enveloped viruses are smaller than enveloped viruses and are much simpler in structure, including an outer protein capsid structure that encapsulates the viral genome and nonstructural proteins. Minute virus of mice (MVM) belongs to the Parvovirus family, and is 18-26 nm in diameter. The capsid is organized into a T=1 icosahedral shape and includes 60 copies of three different capsid proteins: VP1 (83 kDa), VP2 (63 kDa), and VP3 (61 kDa). Mammalian Orthoreovirus Type 3 (Reo-3) belongs to the Reovirus family, and ranges in size from 75-85 nm in diameter. Reo-3 contains 8 structural proteins organized into two concentric capsids. The inner capsid is composed of λ1 (142 kDa), λ3 (142 kDa), μ2 (83 kDa), and σ2 (47 kDa) proteins organized into a T=2 pseudo icosahedral shape. The outer capsid is composed of λ2 (144 kDa), μ1 (76 kDa), σ3 (41 kDa), and σ1 (49 kDa) proteins organized into a T=131 icosahedron.

Enveloped viruses are surrounded by a viral envelope, i.e., a lipid bilayer decorated with viral envelope proteins, and are larger than non-enveloped viruses and more complex in structure. The murine leukemia virus (MuL V) is a spherical gammaretrovirus 100-125 nm in diameter. The fully assembled virion includes a capsid made of Gag proteins, that are further surrounded by a viral envelope including the Env protein and other cell membrane proteins. The Herpes Simplex Virus Type 1 (HSV-1) is a highly complex enveloped virus that is spherical or pleomorphic in shape and measures 150-200 nm in diameter. HSV-1 is constructed of 4 main components: the genome core, capsid, tegument, and envelope. The viral envelope is composed of portions of the host cell nuclear membranes, the endoplasmic reticulum, and the plasma membrane, and therefore includes a wide range of host cell membrane proteins and lipid components.

Viral surrogate particles are a noninfectious alternative to live virus, and as a result viral clearance work can be run in-house in a BSL-1 environment, reducing the need for time- and cost-intensive CRO collaboration. At this time, there are no viral surrogates that are close enough mimics of live virus to be used in full-fledged regulatory validation work; this is based on the differences seen in clearance mechanisms between live virus and surrogate particles across various purification unit operations. Without wishing to be bound by theory, two general types of viral surrogates currently exist: viral-like particles (VLPs) and bacteriophage models. VLP-based surrogate development is currently led by Mock V™ Solutions (now a part of Maravai Life Sciences), where MVM mock viral particles (MVM-MVPs) have been generated and retrovirus-like particles (RVLPs) are under development. MVM-MVPs are produced by cloning and expressing MVM capsid protein VP2 in a baculovirus vector system via DH10Bac cells, followed by transfection into sf9 insect cells. VP2 is then purified and allowed to self-assemble into VLPs; the resulting particle is 25.6±1.5 nm in diameter with an isoelectric point (pl) of 5.8, similar to live MVM (18-26 nm diameter, pl of 6.0-6.2). Quantification of MVM-MVPs involves a coupled immuno-sandwich qPCR/ELISA technique, which involves multiple steps and provides a range of detection of 4 log LRV and an LOD of 5 log MVP/mL. This level of sensitivity is not representative of current live virus quantification capability. Mock V™ RVLPs are endogenously produced via a host cell line of choice (to align with the process of interest) and purified from cell culture broth using Protein A affinity chromatography (flowthrough and wash fractions) and size exclusion chromatography. RVLP quantification is flexible in that either qPCR or the immune-sandwich technique can be employed. Four commonly utilized bacteriophages in viral clearance studies are ΦX174, Φ6, PR772, and PP727. These phages are fairly representative of model mammalian viruses with respect to size and isoelectric point and are therefore employed as general model viruses in early phase work. The benefits of working with phages include non-infectivity in mammalian cells (can be used in-house), and rapid phage culture and plaque titer assay quantification through utilization of E. coli (ΦX174, PR772), P. aeruginosa (PP7), and P. syringae (Φ6) as indicator cells.

### SUMMARY

Aspects of the present disclosure are directed to a system for evaluating viral clearance processes. In some embodiments, the system includes a process solution including a target drug of interest. In some embodiments, the process solution includes a plurality of viral surrogate nanoparticles. In some embodiments, the particles include a core including one or more fluorescent materials and a viral surface-mimicking layer on a surface of the core, wherein the viral surface-mimicking layer physicochemically mimics an external surface of a target live virus equivalent. In some embodiments, the viral surface-mimicking layer includes one or more capsid or capsid-like proteins of the target live virus equivalent bound to the core, embedded in a lipid bilayer attached to the core, or combinations thereof. In some embodiments, the system includes a membrane separator in fluid communication with the process solution. In some embodiments, the membrane separator includes a polycarbonate substrate having a plurality of pores extending therethrough and one or more surfaces on the substrate, the surfaces being configured to bind the viral surface-mimicking layer of the viral surrogate nanoparticles. In some embodiments, the system includes a fluorescence emission spectrophotometer positioned to identify a fluorescent signal from viral surrogate nanoparticles, e.g., adsorbed onto, the membrane separator, e.g., following a filtration of the particle-containing process solution using the membrane separator.

In some embodiments, the target drug of interest includes an antibody, non-antibody protein, vaccine, nucleic acid product, blood or plasma derivative, or combinations thereof. In some embodiments, the core includes one or more polymers including polystyrene, polystyrene copolymers, or combinations thereof. In some embodiments, the viral surrogate nanoparticles mimic the size, shape, net charge, hydrophobicity, or combinations thereof, of the target live virus equivalent. In some embodiments, the cores include one or more additional chemical treatments to the surface of the core, the additional chemical treatments configured further improve physicochemical mimicking of the target live virus equivalent by the viral surrogate nanoparticles. In some embodiments, the one or more capsid or capsid-like proteins are from a family including Parvoviridae, Reoviridae, Retroviridae, Herpesviridae, or combinations thereof. In some embodiments, the lipid bilayer is attached to the core via one or more lipid anchors. In some embodiments, the core has a diameter between about 20 nm and about 200 nm. In some embodiments, the pore size of the membrane separator is about 50 nm or 200 nm. In some embodiments, the one or more surfaces on the substrate bears a net charge opposite of the viral surrogate nanoparticles or includes one or more moieties that: promote binding of the viral surrogate nanoparticles to the membrane separator; bear a net charge opposite of the viral surrogate nanoparticles; or combinations thereof. In some embodiments, the process solution is produced from a cell culture process, a fermentation process, or combinations thereof. In some embodiments, the one or more capsid or capsid-like proteins cover about 55% of the surface of the core.

Aspects of the present disclosure are directed to a method for evaluating viral clearance of a sample. In some embodiments, the method includes preparing a process solution including a target drug of interest and a plurality of viral surrogate nanoparticles configured to mimic the size, shape, net charge, hydrophobicity, or combinations thereof, of a target live virus equivalent. In some embodiments, the particles include a core including one or more fluorescent materials and a viral surface-mimicking layer on a surface of the core. In some embodiments, the viral surface-mimicking layer physicochemically mimics an external surface of a target live virus equivalent. In some embodiments, the viral surface-mimicking layer includes one or more capsid or capsid-like proteins of the target live virus equivalent are directly associated with: the core; a lipid bilayer attached to the core, or combinations thereof.

In some embodiments, the method includes treating at least a portion of the process solution with one or more purification processes to form a product process solution, the one or more purification processes configured to remove one or more impurities including target live virus particles. In some embodiments, the method includes contacting the product process solution with a membrane separator. In some embodiments, the membrane separator includes a polycarbonate substrate having a plurality of pores extending therethrough and one or more surfaces on the substrate, the surfaces being configured to bind the viral surface-mimicking layer of the viral surrogate nanoparticles. In some embodiments, the method includes performing a solid-phase fluorescence intensity measurement of the particles captured on the membrane separator from the product process solution to quantify the concentration of viral surrogate nanoparticles in the product process solution. In some embodiments, the method includes retaining at least a portion of the process solution as a load process solution. In some embodiments, the method includes contacting the load process solution with a membrane separator. In some embodiments, the method includes performing a solid-phase fluorescence intensity measurement of the particles captured on the membrane separator from the load process solution to quantify the concentration of viral surrogate nanoparticles in the load process solution. In some embodiments, the method includes determining a log reduction value (LRV) of the one or more purification processes by subtracting the common log value of the concentration of viral surrogate nanoparticles in the product process solution from the common log value of the concentration of viral surrogate nanoparticles in the load process solution.

In some embodiments, the target live virus belongs to a family including Parvoviridae, Reoviridae, Retroviridae, Herpesviridae, or combinations thereof. In some embodiments, the target drug of interest includes an antibody, non-antibody protein, vaccine, nucleic acid product, blood or plasma derivative, or combinations thereof.

Aspects of the present disclosure are directed to a method for evaluating viral clearance of a sample. In some embodiments, the method includes modifying a plurality of nanoparticle cores that include one or more fluorescent materials to include a viral surface-mimicking layer. In some embodiments, the viral surface-mimicking layer physicochemically mimics the external surface of a target live virus equivalent, wherein the viral surface-mimicking layer includes one or more capsid or capsid-like proteins of the target live virus equivalent directly associated with: the core; a lipid bilayer attached to the core via one or more lipid anchors, or combinations thereof. In some embodiments, the method includes modifying at least a first membrane and a second membrane in a dead-end flow nanofiltration membrane separator with one or more surfaces configured to bind the modified nanoparticle cores. In some embodiments, the membrane includes a polycarbonate substrate having a plurality of pores extending therethrough; wherein at least one of the surfaces include polyethyleneimine (PEI); and wherein the one or more surfaces are on the substrate and: bears a net charge opposite of the modified nanoparticle cores; or includes one or more moieties that: promote binding of the modified nanoparticle cores to the membrane separator; bear a net charge opposite of the modified nanoparticle cores; or combinations thereof.

In some embodiments, the method includes obtaining a sample from a cell culture process, a fermentation process, or combinations thereof, the sample including a target drug of interest. In some embodiments, the method includes performing a viral spiking study on the sample. In some embodiments, the viral spiking study includes administering a concentration of the modified nanoparticle cores to the sample to form a process solution; retaining at least a portion of the process solution as a load process solution; performing one or more purification processes on an amount of remaining process solution to form a product process solution; and quantifying modified nanoparticle cores in the load process solution and product process solution. In some embodiments, quantifying modified nanoparticle cores includes filtering an established volume of the product process solution across the first membrane; filtering an established volume of the load process solution across the second membrane; performing a fluorescence intensity measurement of the modified nanoparticle cores captured on the first and second membranes; performing a baseline decomposition of the fluorescence intensity measurements; applying a standard curve to the baseline decomposed fluorescence data to calculate particle solution concentration; and calculating an LRV by subtracting the common log value of the concentration of modified nanoparticle cores in the product process solution from the common log value of the concentration of modified nanoparticle cores in the load process solution. In some embodiments, the target live virus belongs to a family including Parvoviridae, Reoviridae, Retroviridae, Herpesviridae, or combinations thereof. In some embodiments, the target drug of interest includes an antibody, non-antibody protein, viral vector, nucleic acid product, blood or plasma derivative, or combinations thereof.

In some embodiments, modifying the plurality of nanoparticle cores includes covalently binding the one or more capsid or capsid-like proteins using 1-ethyl-3-(3-dimethylaminopropyl)carbodiimide-N-hydroxysuccinimide conjugation chemistry to the nanoparticle cores. In some embodiments, modifying the plurality of nanoparticle cores includes binding primary amine-modified head group lipid anchors to the nanoparticle cores using 1-ethyl-3-(3-dimethylaminopropyl)carbodiimide-N-hydroxysuccinimide conjugation chemistry; extruding liposomes including a lipid composition from the target live virus equivalent and membrane proteins including the one or more capsid or capsid-like proteins; and incubating the lipid anchor-functionalized nanoparticle cores with the extruded liposomes, resulting in liposome self-assembly into a bilayer on the cores.

### BRIEF DESCRIPTION OF DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic representation of a system for quantifying virus concentration of a viral clearance sample according to some embodiments of the present disclosure;

FIG. 2A is a schematic representation of viral surrogate nanoparticles according to some embodiments of the present disclosure;

FIG. 2B is a schematic representation of viral surrogate nanoparticles according to some embodiments of the present disclosure;

FIG. 3 is a schematic representation of membrane separators according to some embodiments of the present disclosure;

FIG. 4A is a chart of a method for evaluating viral clearance of a sample according to some embodiments of the present disclosure;

FIG. 4B is a chart of a method for performing a viral spiking study according to some embodiments of the present disclosure;

FIG. 5 is a chart of a method for quantifying concentration of viral particles in a process solution following viral clearance according to some embodiments of the present disclosure;

FIG. 6 is a schematic representation of an exemplary 1-ethyl-3-(3-dimethylaminopropyl)carbodiimide-N-hydroxysuccinimide conjugation chemistry used to attach capsid proteins to viral surrogate nanoparticles consistent with embodiments of the present disclosure;

FIG. 7A portrays graphs and tabulated dynamic light scattering (DLS) data summarizing size distribution information from a colloidal stability study performed using 20 nm viral surrogate nanoparticles consistent with embodiments of the present disclosure;

FIG. 7B portrays graphs and tabulated DLS data summarizing size distribution information from an additional colloidal stability study performed using 20 nm viral surrogate nanoparticles consistent with embodiments of the present disclosure;

FIG. 8A is a set of output figures from fluorescence baseline decomposition MATLAB code that presents the baseline decomposition output for samples of viral surrogate nanoparticles consistent with embodiments of the present disclosure;

FIG. 8B is an output figure from fluorescence baseline decomposition MATLAB code that presents a standard curve utilized to convert sample fluorescence intensity into concentration of viral surrogate nanoparticles consistent with embodiments of the present disclosure;

FIGS. 9A-9D are graphs portraying mass accumulated versus time for ultrapure water filtrations across bare 15 nm, 30 nm, 50 nm, and 200 nm pore size polycarbonate track-etched membranes consistent with embodiments of the present disclosure at varying differential pressures;

FIGS. 10A-10D are graphs portraying mass accumulated versus time for ultrapure water filtrations and nanoparticle filtrations at various concentrations across bare 15 nm, 30 nm, 50 nm, and 200 nm pore size polycarbonate track-etched membranes according to some embodiments of the present disclosure at varying differential pressures;

FIG. 11 portrays scanning electron microscopy (SEM) images comparing inconsistent particle adsorption on bare 15 nm, 30 nm, and 50 nm polycarbonate track-etched membranes according to some embodiments of the present disclosure;

FIG. 12 portrays SEM images showing consistent and effective particle adsorption on 200 nm pore size polyethyleneimine (PEI) modified polycarbonate track-etched membranes consistent with embodiments of the present disclosure, operated at 15 psid;

FIG. 13 is a graph portraying an exemplary combined standard curve for a 200 nm polycarbonate track-etched membrane modified with PEI according to some embodiments of the present disclosure, relating volume filtered and fluorescence intensity to viral surrogate nanoparticles adsorbed, nanoparticle fluorescence limits of detection, and process solution nanoparticle concentration;

FIGS. 14A-14B are graphs portraying fluorescence intensity versus viral surrogate nanoparticles concentration standard curves for 100 mL filtrations of 20 nm and 100 nm particles across a PEI-modified, 200 nm nominal pore size polycarbonate track-etched membranes according to some embodiments of the present disclosure; and

FIG. 15 is a graph showing a fluorescence intensity versus viral surrogate nanoparticle concentration standard curve for 100 mL filtrations of 100 nm particles across a PEI-modified, 200 nm nominal diameter polycarbonate track-etched membrane, evaluating filtration performance in high salt environments and with nanoparticles consistent with embodiments of the present disclosure.

### DETAILED DESCRIPTION

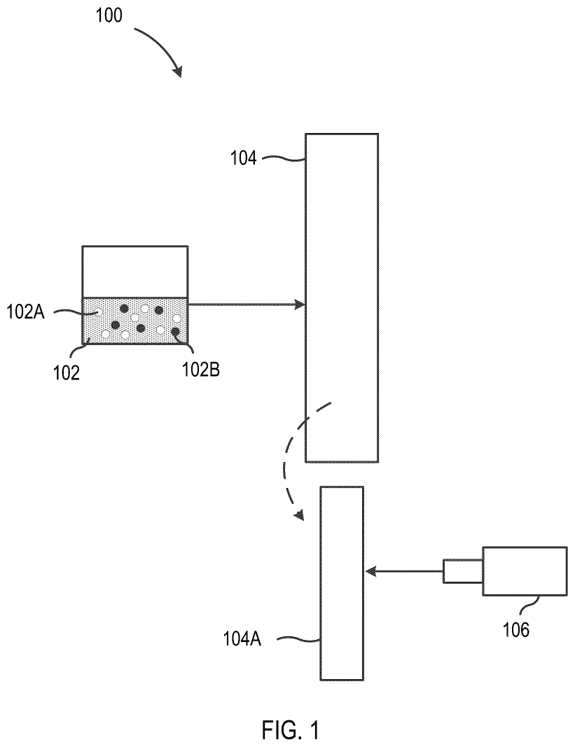

Referring now to FIG. 1, some embodiments of the present disclosure are directed to a system 100 for quantifying virus concentration of a viral clearance sample. As will be discussed in greater detail below, in some embodiments, the viral clearance processes involve introducing a known amount and/or concentration of viral surrogate nanoparticles representative of a target live virus into a sample including a target product of interest, e.g., a drug. An aliquot of the sample is collected from the load process solution, while at least an amount of the remaining load process solution sample is subjected to one or more downstream purification processes of interest to form the product process solution. In some embodiments, the purification processes include chromatography, filtration, ultrafiltration, precipitation, centrifugation, viral inactivation techniques, extraction, or combinations thereof. The amount or concentration of persisting viral surrogate nanoparticles in the product process solution is determined. In some embodiments, determining the amount of viral surrogate nanoparticles includes enzyme-linked immunosorbent assay (ELISA), nanoimaging, fluorescence, enzymatic, microscopy, spectrophotometry, electron microscopy (EM), western blot analyses techniques, or combinations thereof. In some embodiments, determining the amount of viral surrogate nanoparticles includes a dead-end flow nanofiltration to capture viral surrogate nanoparticles via a membrane surface, followed by consecutive solid-phase fluorescence intensity measurement and analysis of the resulting raw fluorescence data to extract the pure viral surrogate nanoparticles fluorescence signal for conversion to a viral surrogate nanoparticle concentration, e.g., via a standard curve as will be discussed in greater detail below. In some embodiments, a virus logarithmic reduction value is then calculated as the difference in the logarithm of the viral surrogate nanoparticles amount or concentration in the load processes solution compared to that in the product-containing efflux from the relevant downstream purification process or processes, i.e., the product process solution.

Referring again to FIG. 1, system 100 includes a process solution 102. As discussed above, process solution 102 includes a target drug of interest 102A. In some embodiments, target drug of interest 102A is a biologic drug or combination of biological drugs. In some embodiments, target drug of interest 102A includes an antibody, non-antibody protein, viral vector, nucleic acid product, blood or plasma derivative, or combinations thereof. In some embodiments, target drug of interest 102A is produced from a cell culture process, a fermentation process, or combinations thereof. In some embodiments, this production process utilizes human cells, animal cells, plant cells, insect cells, hybridomas cells, yeast cell, bacterial cells, or combinations thereof.

In some embodiments, process solution 102 includes a plurality of viral surrogate nanoparticles 102B. In some embodiments, viral surrogate nanoparticles 102B mimic the size, shape, net charge, surface composition, hydrophobicity, or combinations thereof, of a target live virus equivalent, e.g., relevant model viruses applied in viral clearance validation. In some embodiments, viral surrogate nanoparticles 102B are configured to mimic one or more non-enveloped viruses. In some embodiments, viral surrogate nanoparticles 102B are configured to mimic one or more enveloped viruses.

In some embodiments, process solution 102 is produced by providing an amount or a concentration of viral surrogate nanoparticles 102B to the product of a cell culture process, a fermentation process, etc. discussed above with respect to producing the target drug of interest. The viral surrogate nanoparticles 102B consistent with some embodiments of the present disclosure physicochemically mimic various non-enveloped or enveloped viruses, and thus can advantageously be applied to viral clearance validation as an alternative to live virus, or more generally to biological studies concerning cellular uptake, accumulation, and interfacial adsorption kinetics.

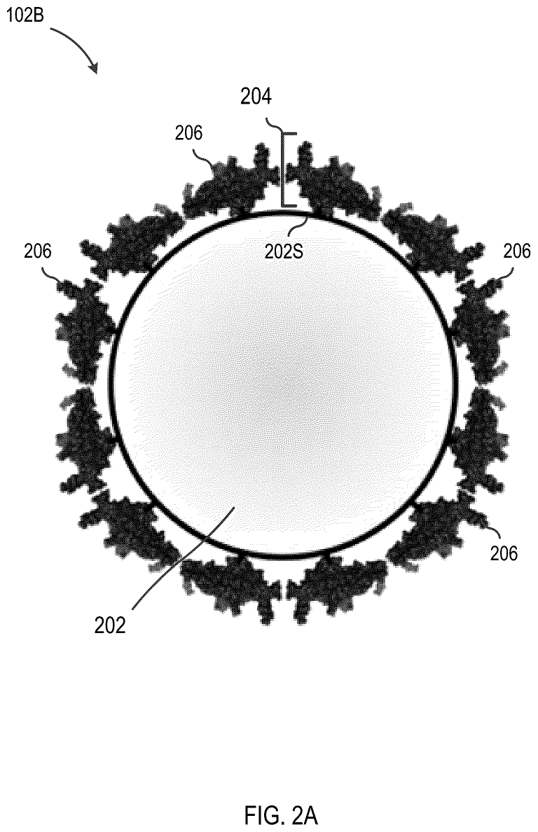

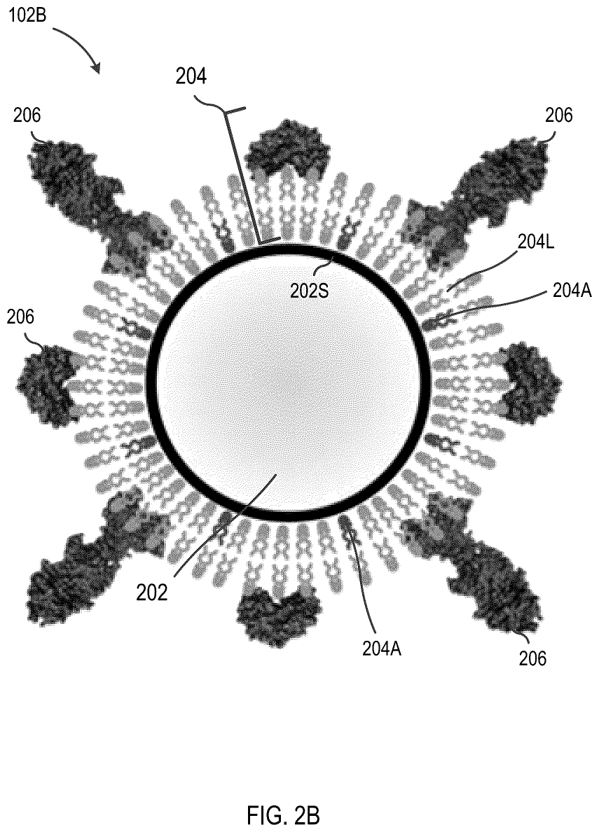

Referring now to FIG. 2A, in some embodiments, viral surrogate nanoparticles 102B include a nanoparticle core 202. In some embodiments, core 202 is made of any suitable material that can be functionalized, as discussed in greater detail below, so as to mimic a target live virus equivalent. In some embodiments, core 202 is composed of one or more metals, polymers, biopolymers, proteins, or combinations thereof. In some embodiments, core 202 is rigid. In some embodiments, core 202 includes one or more materials that can be activated to provide available carboxylate groups on the surface thereof. In some embodiments, core 202 includes one or more polymers including polystyrene, polystyrene copolymers, or combinations thereof. In some embodiments, core 202 includes one or more fluorescent materials.

In some embodiments, viral surrogate nanoparticles 102B include a viral surface-mimicking layer 204 on a surface 202S of core 202. In some embodiments, viral surface-mimicking layer 204 physicochemically mimics an external surface of a target live virus equivalent. In some embodiments, viral surface-mimicking layer 204 fully surrounds core 202. In some embodiments, viral surface-mimicking layer 204 partially surrounds core 202. In some embodiments, viral surface-mimicking layer 204 fully covers surface 202S. In some embodiments, viral surface-mimicking layer 204 partially covers surface 202S.

In some embodiments, viral surface-mimicking layer 204 includes proteins 206. In some embodiments, viral surface-mimicking layer 204 includes one or more capsid or capsid-like proteins 206 of the target live virus equivalent. In some embodiments, at least some of proteins 206 have the same sequence or substantially the same sequence as a naturally occurring protein, e.g., capsid protein. In some embodiments, at least some of proteins 206 are recombinant versions of a naturally occurring proteins. In some embodiments, proteins 206 are produced in a bacteria, yeast, plant, insect cell, animal or human cell.

In some embodiments, at least one of proteins 206 are functional equivalent or substantially functionally equivalent to a naturally occurring proteins when bound to viral surrogate nanoparticles 102B, as will be discussed in greater detail below. In some embodiments, proteins 206 are from a family including Parvoviridae, Reoviridae, Retroviridae, Herpesviridae, or combinations thereof. In some embodiments, proteins 206 include VP1, VP2, or VP3 from protoparvovirus; μ1, σ1, σ3, or λ2 from reovirus; Env from gammaretrovirus; and gB, gD, gH, or gL from simplexvirus, recombinant functional equivalents thereof, or combinations thereof.

Referring specifically to FIG. 2B, in some embodiments, viral surrogate nanoparticles 102B are constructed consistent with enveloped live virus particles at least via the inclusion of lipid layer in viral surface-mimicking layer 204. In some embodiments, the lipid layer is a lipid bilayer 204L. In some embodiments, lipid bilayer 204L is attached to core 202 via one or more lipid anchors 204A. In some embodiments, the composition of lipid bilayer 204L, including the presence of any proteins 206 contained therein, mimics the surface composition and/or associated physicochemical characteristics of a target enveloped live virus equivalent.

In some embodiments, proteins 206 are attached to core 202, lipid bilayer 204L (see FIG. 2B), or combinations thereof. In some embodiments, proteins 206 are attached to core 202 via a covalent bond, other non-covalent bonds or forces, or combinations thereof. In some embodiments, proteins 206 are attached to core 202 using 1-ethyl-3-(3-dimethylaminopropyl)carbodiimide-N-hydroxysuccinimide conjugation chemistry. In these exemplary embodiments, surface 202S is provided or functionalized with a plurality of carboxylate groups in solution. In some embodiments, the solution is then spiked with a concentration of 1-ethyl-3-(3-dimethylaminopropyl)carbodiimide to activate the carboxylate ligands on core 202, resulting in an O-acylisourea intermediate. In some embodiments, the solution is then further spiked with a concentration of N-hydroxysuccinimide (NHS) which stabilizes the O-acylisourea intermediate and produces a sulfo-NHS ester. In some embodiments, proteins 206 are added and form peptide bonds with the sulfo-NHS ester via its primary amine.

In exemplary embodiments for viral surrogate nanoparticles 102B mimicking enveloped live virus equivalents, lipid anchors 204A are added and form peptide bonds with the sulfo-NHS ester via its primary amine. In some embodiments, one or more liposomes are then extruded to envelope core 202. In some embodiments, the liposomes have a lipid composition functionally the same or similar to that of the target live virus equivalent. In some embodiments, the liposomes are include one or more membrane proteins from the target live virus equivalent, i.e., proteins 206. In some embodiments, cores 202 functionalized with lipid anchors 204A are incubated with the extruded liposomes. During incubation, the liposomes self-assemble into bilayer 204L on core 202. Without wishing to be bound by theory, because lipid mobility is of low concern for the embodiments of the present disclosure, the bilayers can be tethered to a rigid nanoparticle core, e.g., core 202.

In some embodiments, proteins 206 cover more than about 5%, 10%, 15%, 20%, 25%, 30%, 35%, 40%, 45%, 50%, etc. of surface 202S. In some embodiments, proteins 206 cover about 55% of surface 202S. In some embodiments, proteins 206 are attached via a linker. In some embodiments, lipid anchors 204A are attached via a linker.

In some embodiments, cores 202 include one or more additional chemical treatments to surface 202S. In some embodiments, the additional chemical treatments are configured to further improve physicochemical mimicking of target live virus equivalents by viral surrogate nanoparticles 102B. In some embodiments, at least a first portion of surface 202S is covered by viral surface-mimicking layer 204, and at least a second portion includes the one or more additional chemical treatments. In some embodiments, the one or more additional chemical treatments include functionalizing surface 202S with one or more moieties.

In some embodiments, cores 202 are sized such that, upon functionalization, e.g., with viral surface-mimicking layer 204, viral surrogate nanoparticles 102B are the same size or substantially the same size as the target virus equivalent. In some embodiments, core 202 has a diameter between about 20 nm and about 200 nm. In some embodiments, core 202 has a diameter between about 40 nm to about 100 nm. In some embodiments, core 202 has a diameter between about 60 nm to about 80 nm. In some embodiments, the combination of core 202 with viral surface-mimicking layer 204 has a diameter between about 20 nm and about 200 nm. In some embodiments, the combination of core 202 with viral surface-mimicking layer 204 has a diameter between about 40 nm to about 100 nm. In some embodiments, the combination of core 202 with viral surface-mimicking layer 204 has a diameter between about 60 nm to about 80 nm. In some embodiments, cores 202 are shaped such that, upon functionalization, e.g., with viral surface-mimicking layer 204, viral surrogate nanoparticles 102B are the same shape or substantially the same shape as the target virus equivalent.

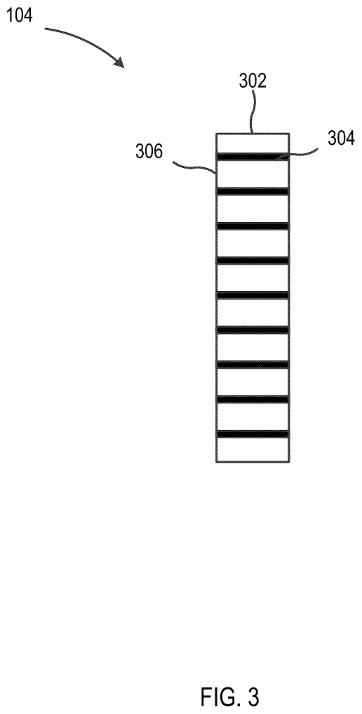

Referring again to FIG. 1, in some embodiments, system 100 includes a membrane separator 104. In some embodiments, membrane separator 104 is in fluid communication with process solution 102. Referring now to FIG. 3, in some embodiments, membrane separator 104 includes a substrate 302. In some embodiments, substrate 302 includes polycarbonate. In some embodiments, substrate 302 includes a plurality of pores 304 extending therethrough.

In some embodiments, substrate 302 includes one or more surfaces 306 thereon. In some embodiments, surface 306 (including, in some embodiments, the interior of pores 304) is configured to attract and/or bind viral surface-mimicking layer 204 from viral surrogate nanoparticles 102B. In some embodiments, surfaces 306 bear a net charge opposite that of viral surrogate nanoparticles 102B. In some embodiments, one or more moieties are bound to surfaces 306 that promote binding of viral surrogate nanoparticles 102B to membrane separator 104, bear a net charge opposite of the nanoparticles, or combinations thereof. In some embodiments, surface 306 is functionalized with one or more molecules or macromolecules to promote viral surrogate nanoparticle 102B affinity, for example, by exploiting electrostatic, hydrogen bonding, hydrophobic and/or affinity interactions to enhance the capture of the nanoparticles by surface 306 and provide consistent membrane surface adsorption for subsequent quantification. In some embodiments, surfaces 306 include polyethyleneimine (PEI).

As used herein, the term “pore size” is used to refer to the functional size of pores 304 after membrane separator 104 has been configured and/or modified to bind viral surrogate nanoparticles 102B, e.g., after one or more moieties have been bound to surfaces 306 on substrate 302. In some embodiments, the pore size of membrane separator 104 is about 50 nm. In some embodiments, the pore size of membrane separator 104 is about 200 nm.

Referring again to FIG. 1, in some embodiments, system 100 includes a fluorescence emission spectrophotometer 106. In some embodiments, spectrophotometer 106 is positioned to identify a fluorescent signal from viral surrogate nanoparticles 102B from membrane separator 104. In some embodiments, spectrophotometer 106 is positioned to identify a fluorescent signal from viral surrogate nanoparticles 102B from membranes within membrane separator 104, that have been removed from membrane separator 104, or combinations thereof. In some embodiments, a membrane 104A is removable from membrane separator 104 for imaging viral surrogate nanoparticles 102B thereon, e.g., via spectrophotometer 106.

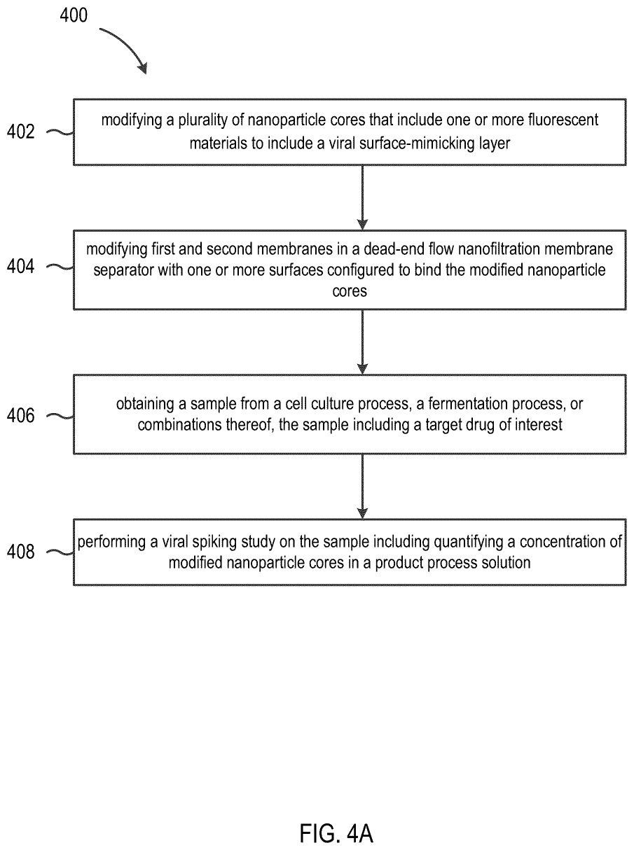

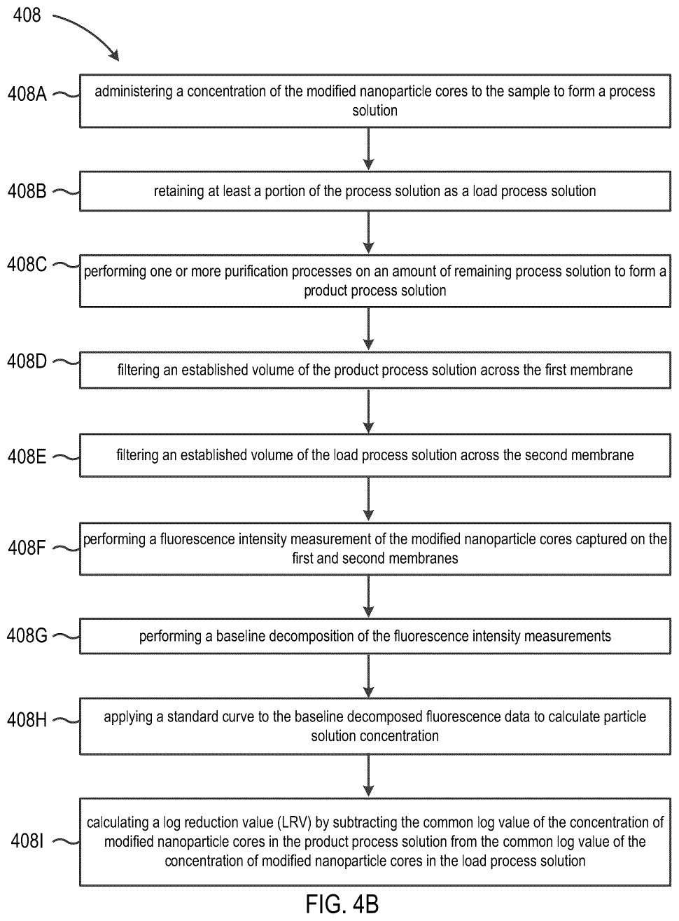

Referring now to FIG. 4A, some embodiments of the present disclosure are directed to a method 400A for evaluating viral clearance of a sample. In some embodiments, at 402, a plurality of nanoparticle cores are modified to include a viral surface-mimicking layer. In some embodiments, the cores are modified 402 to mimic the size, shape, net charge, hydrophobicity, or combinations thereof, of a target live virus equivalent. As discussed above, in some embodiments, the nanoparticle cores include one or more polymers such that the surface can be activated to provide available carboxylate groups. In some embodiments, nanoparticle cores include one or more polymers including polystyrene, polystyrene copolymers, or combinations thereof. In some embodiments, the nanoparticles cores include one or more fluorescent materials. In some embodiments, the viral surface-mimicking layer physicochemically mimics the external surface of a target live virus equivalent. In some embodiments, the target live virus belongs to a family including Parvoviridae, Reoviridae, Retroviridae, Herpesviridae, or combinations thereof. In some embodiments, the viral surface-mimicking layer includes one or more capsid or capsid-like proteins of a target live virus equivalent. In some embodiments, the proteins are bound to one of the core; embedded in a lipid bilayer attached to the core via one or more lipid anchors, or combinations thereof. In some embodiments, as discussed above, one or more additional treatments are made to the core to further improve physicochemical mimicking of target live virus equivalents. In some embodiments, a first portion of the core is modified with the binding of proteins to the surface thereof, and a second portion of the core is modified via the one or more additional treatments.

In some embodiments, modifying 402 the plurality of nanoparticle cores includes covalently binding the one or more capsid or capsid-like proteins to the nanoparticle cores using 1-ethyl-3-(3-dimethylaminopropyl)carbodiimide-N-hydroxysuccinimide conjugation chemistry. In some embodiments, modifying 402 the plurality of nanoparticle cores includes binding primary amine-modified head group lipid anchors to the nanoparticle cores using 1-ethyl-3-(3-dimethylaminopropyl)carbodiimide-N-hydroxysuccinimide conjugation chemistry; extruding liposomes including a lipid composition from the target live virus equivalent and membrane proteins including the one or more capsid or capsid-like proteins; and incubating the lipid anchor-functionalized nanoparticle cores with the extruded liposomes, resulting in liposome self-assembly into a bilayer on the cores.

Still referring to FIG. 4A, in some embodiments, at 404, a membrane is modified. In some embodiments, the membrane is included in a dead-end flow nanofiltration membrane separator. In some embodiments, at least a first and a second membrane are modified at 404, so that various process solutions can be administered to separate, clean membranes. In some embodiments, the membrane is modified 404 to include one or more surfaces configured to bind the modified nanoparticle cores. As discussed above, in some embodiments, the membrane includes a polycarbonate substrate having a plurality of pores extending therethrough. In some embodiments, the one or more surfaces are on the substrate bears a net charge opposite of the modified nanoparticle cores; or includes one or more moieties that promote binding of the modified nanoparticle cores to the membrane separator; bear a net charge opposite of the modified nanoparticle cores; or combinations thereof. In some embodiments, the one or more surfaces include PEI.

Referring again to FIG. 4A, in some embodiments, at 406, a sample is obtained. In some embodiments, the sample includes a target drug of interest. In some embodiments, the target drug of interest includes an antibody, non-antibody protein, vaccine, nucleic acid product, blood or plasma derivative, or combinations thereof. In some embodiments, the target drug of interest is produced from a cell culture process, a fermentation process, or combinations thereof. In some embodiments, the target drug of interest is obtained 406 from the cell culture process, fermentation process, etc.

In some embodiments, at 408, a viral spiking study is performed on the sample. Referring now to FIG. 4B, in some embodiments, viral spiking study 408 includes administering 408A a concentration of the modified nanoparticle cores to the sample to form a process solution. In some embodiments, a plurality of different modified nanoparticle cores are administered at step 408A, i.e., modified nanoparticle cores mimicking two or more target live virus equivalents. In some embodiments, the two or more different modified nanoparticle cores include different fluorescent materials. In some embodiments, the two or more different modified nanoparticle cores are provided 408A simultaneously or substantially simultaneously. In some embodiments, the two or more different modified nanoparticle cores are provided 408A are provided sequentially. In some embodiments, a concentration of the target live virus equivalents are also administered at step 408A. In some embodiments, at 408B at least a portion of the process solution is retained as a load process solution. In some embodiments, at 408C, one or more purification processes is performed on an amount of remaining process solution to form a product process solution. As discussed above, in some embodiments, the purification processes include a chromatography, filtration, ultrafiltration, precipitation, centrifugation, viral inactivation technique, extraction, or combinations thereof. In some embodiments, modified nanoparticle cores in the load process solution and product process solution are then quantified. In some embodiments, this quantification step includes, at 408D, filtering an established volume of the product process solution across the first membrane. In some embodiments, at 408E, an established volume of the load process solution is filtered across the second membrane. In some embodiments, the modified first and second membranes are included in a dead-end flow nanofiltration membrane separator. In some embodiments, this analytical process including dead-end flow is a batch-processing unit operation, semi-batch unit operation, continuous unit operation, or combinations thereof. In some embodiments, at 408F, fluorescence intensity measurements of the modified nanoparticle cores captured on the first and second membranes are performed. In some embodiments, at 408G, baseline decomposition of the fluorescence intensity measurements are performed. In some embodiments, at 408H, a standard curve is applied to the baseline decomposed fluorescence data to calculate particle solution concentration, as will be discussed in greater detail below. In some embodiments, at 408I, a log reduction value (LRV) is calculated by subtracting the common log value of the concentration of modified nanoparticle cores in the product process solution from the common log value of the concentration of modified nanoparticle cores in the load process solution.

In some embodiments, at 408D, an established volume of the product process solution is filtered across a membrane modified at step 404. In some embodiments, at 408E, a fluorescence intensity measurement of the modified nanoparticle cores captured on the membrane from the product process solution is identified.

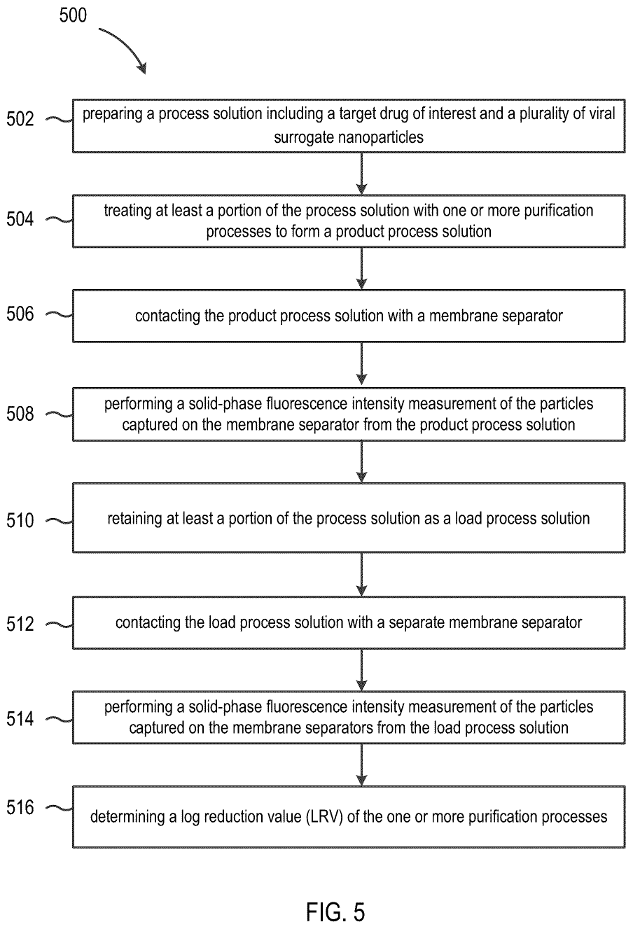

Referring now to FIG. 5, some embodiments of the present disclosure are directed to a method 500 for quantifying concentration of viral particles in a process solution following viral clearance evaluation. In some embodiments, at 502, a process solution is prepared. In some embodiments, the process solution includes a target drug of interest and a plurality of viral surrogate nanoparticles configured to mimic the size, shape, net charge, hydrophobicity, or combinations thereof, of a target live virus equivalent. As discussed above, in some embodiments, the target drug of interest includes an antibody, non-antibody protein, vaccine, nucleic acid product, blood or plasma derivative, or combinations thereof. In some embodiments, the target live virus belongs to a family including Parvoviridae, Reoviridae, Retroviridae, Herpesviridae, or combinations thereof. In some embodiments, the viral surrogate nanoparticles include a core including one or more fluorescent materials; and a viral surface-mimicking layer on a surface of the core. In some embodiments, the viral surface-mimicking layer physicochemically mimics an external surface of a target live virus equivalent. In some embodiments, the viral surface-mimicking layer includes one or more capsid or capsid-like proteins of the target live virus equivalent bound to the core; embedded in a lipid bilayer attached to the core, or combinations thereof.

Still referring to FIG. 5, in some embodiments, at 504, at least a portion of the process solution is treated with one or more purification processes to form a product process solution. The one or more purification processes are configured to remove one or more impurities including target live virus particles. In some embodiments, at 506, the product process solution is contacted with a membrane separator, e.g., a first membrane. In some embodiments, the membrane separator includes a polycarbonate substrate having a plurality of pores extending therethrough. In some embodiments, the membrane separator includes one or more surfaces on the substrate, the surfaces being configured to bind the viral surface-mimicking layer of the viral surrogate nanoparticles.

At 508, a solid-phase fluorescence intensity measurement of the particles captured on the membrane separator from the product process solution is performed to quantify the concentration of viral surrogate nanoparticles in the product process solution after the desired filtration volume has been met.

At 510, at least a portion of the process solution is retained as a load process solution. At 512, the load process solution is contacted with a membrane separator, e.g., a second membrane. In some embodiments, the second membrane is a new, clean membrane. At 514, a solid-phase fluorescence intensity measurement of the particles captured on the membrane separator from the load process solution is performed to quantify the concentration of viral surrogate nanoparticles in the load process solution. In some embodiments, a baseline decomposition of the fluorescence intensity measurements is performed. In some embodiments, a standard curve is applied to the baseline decomposed fluorescence data to calculate particle solution concentration. At 516, an LRV of the one or more purification processes is determined by subtracting the common log value of the concentration of viral surrogate nanoparticles in the product process solution from the common log value of the concentration of viral surrogate nanoparticles in the load process solution.

In exemplary embodiments with a low viral surrogate nanoparticle concentrations, the limit of detection of the assay can be lowered accordingly by increasing the volume of solution filtered, by using a smaller pore size membrane for capture, etc., or combinations thereof. This could allow the user to achieve limits of detection representative of live virus spiking studies, i.e., plaque titer assays.

### EXAMPLES

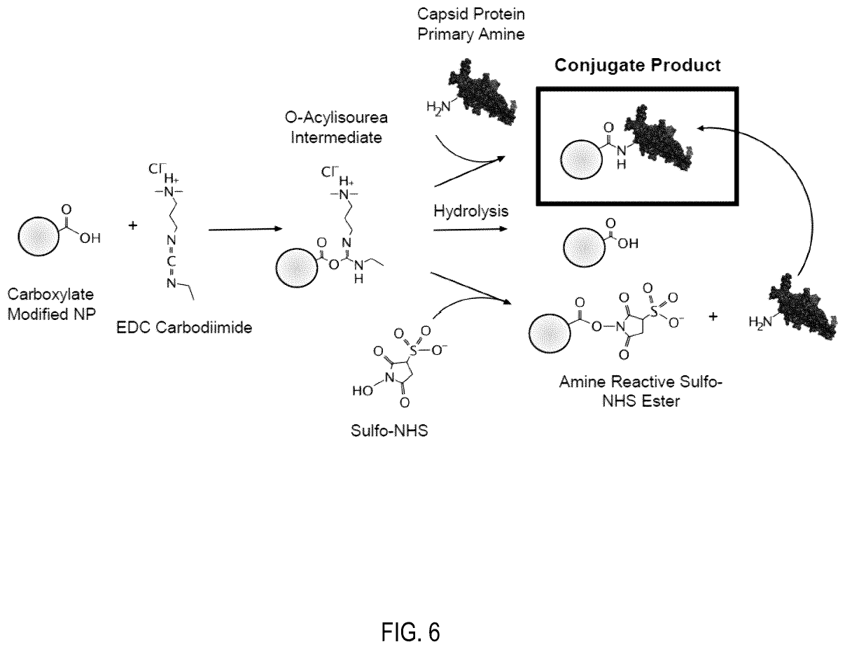

Referring now to FIG. 6, in some exemplary embodiments, to mimic the outside of a non-enveloped virus, the surface of fluorescent viral surrogate nanoparticle cores were covalently modified with the target live virus's outer capsid proteins using 1-ethyl-3-(3-dimethylaminopropyl)carbodiimide-N-hydroxysuccinimide conjugation chemistry (EDC/NHS). Working with this type of chemistry is favorable, as it is effective in reaction environments that maintain nanoparticle colloidal stability and allow for easy application to biologic drug processes. The EDC/NHS reaction is also favorable because it need not involve a linker arm, thus bond lengths between the capsid protein and base particle are not artificially extended, promoting rigid coupling rather than floppy coupling.

To prepare a stock solution of 20 nm non-enveloped viral surrogate nanoparticles, EDC and NHS stock solutions were first prepared in 0.2 μm filtered ultrapure water. EDC/NHS stocks were diluted to 0.2 mM in a volume at least 25% of the reaction volume (e.g., reaction volume (RV)=1 mL, 0.2 mM EDC/NHS stocks were at least 250 μL each). Carboxylate-functionalized nanoparticles (NPs) were activated at 1 ng/ml in 23.6 mM MES, pH 6.5, and 0.05 mM EDC/NHS. This was done by diluting [2.8% RV] μL of stock NP solution into [47.2% RV] μL 50 mM MES, pH 6.5. [25% RV] μL 0.2 mM EDC was added to the nanoparticle solution slowly under constant mixing to prevent pockets of nucleation of aggregated particles. Without wishing to be bound by theory, EDC neutralizes the carboxyl ligand's negative charge upon activation, decreasing net electrostatic forces on the particle surface and allowing hydrophobic forces to dominate and induce aggregation. 0.2 mM NHS was added following the EDC in the same fashion, slowly and under constant mixing, to further prevent aggregation. The solution was then incubated for 30 minutes at ambient conditions under constant mixing (−60 rpm on orbital shaker). Following nanoparticle activation, excess EDC and NHS were removed from the reaction via centrifugal filtration using 50 kDa MWCO centrifugal filter units. Between each cycle, activated NPs were re-suspended to the same volume in fresh 50 mM MES, pH 6.5. After the last cycle, cores were re-suspended to 50% starting volume of 50 mM MES, pH 6.5 to achieve roughly 2 ng/ml cores ([50% RV] μL).

Following this, stock viral capsid protein solution was diluted in 0.2 μm filtered ultrapure water to the desired concentration, to obtain the desired capsid protein:core number ratio. Capsid protein was added to the core solution slowly and under constant mixing. The solution was then incubated for 5 hours at ambient conditions under constant mixing (60 rpm on orbital shaker). Following this, excess unbound capsid protein was removed via centrifugal filtration using 1000 kDa MWCO centrifugal filter units. Before each cycle and after the final cycle, viral surrogate nanoparticles were suspended to the same volume in fresh 50 mM MES, pH 6.5. To assure minimal aggregate formation following synthesis, viral surrogate nanoparticle size and shape distributions were measured via dynamic light scattering (DLS) and scanning electron microscopy (SEM).

Enveloped viruses are much more complex in structure relative to non-enveloped viruses, including 4 main components: the genome core, capsid, tegument, and envelope. As discussed above, lipid mobility is not of particular concern in this case, as a goal of the enveloped viral surrogate nanoparticles is to mimic the viral envelope composition, general size and morphology of the viral particle, isoelectric point, etc.

In exemplary embodiments of the present disclosure, lipids are deposited onto the carboxylate modified viral surrogate nanoparticles core surface. In some embodiments, formulating an enveloped fluorescent viral surrogate nanoparticle core includes: functionalizing a core or substrate with a lipid anchor (this could be done a variety of ways and may depend on the type of substrate utilized, e.g., covalently modifying the surface of carboxylate functionalized nanoparticles with primary amine modified head group lipid anchors using EDC/NHS chemistry); extruding liposomes including representative lipid compositions and membrane proteins; incubating lipid anchor-functionalized substrates with extruded liposomes, resulting in liposome self-assembly into a bilayer on the substrate via rolling/spreading mechanisms through interaction with the lipid anchors. Following synthesis, size and of enveloped viral surrogate nanoparticles were measured via DLS.

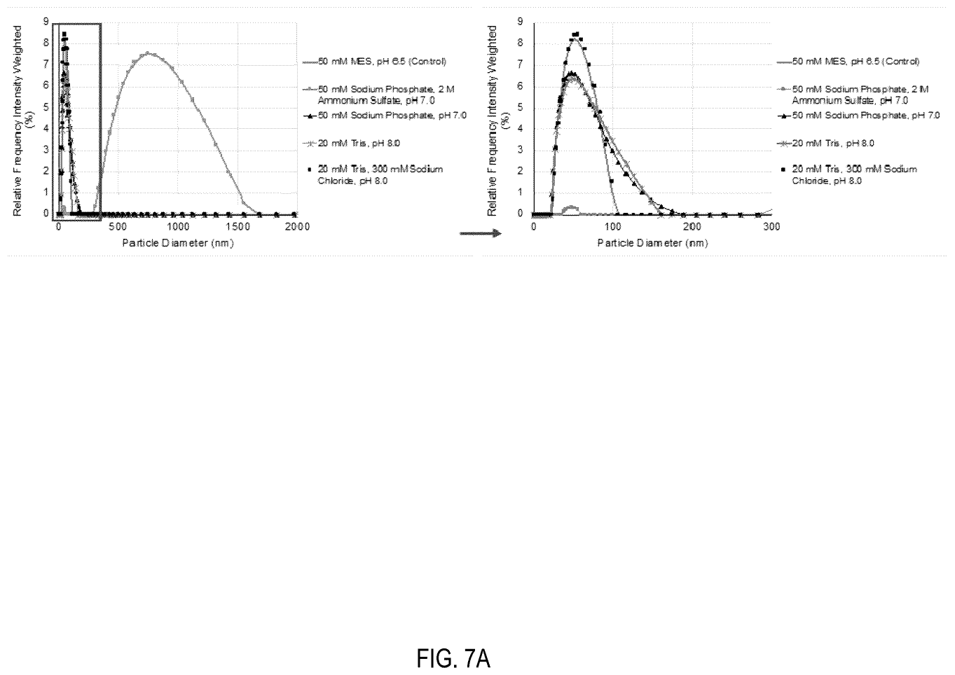

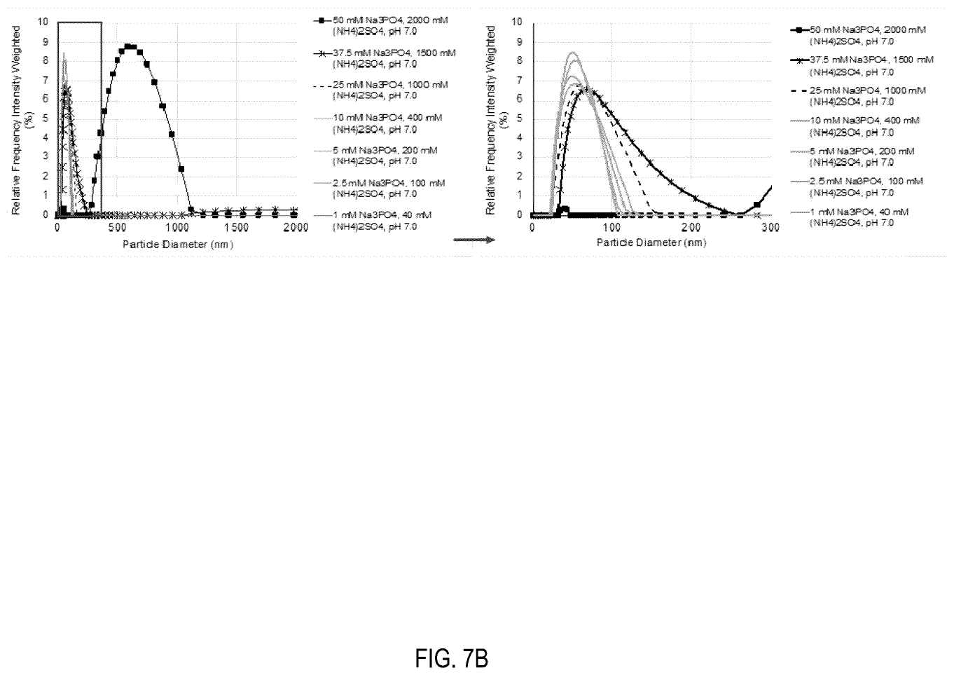

Referring now to FIGS. 7A and 7B, the colloidal stability of viral surrogate nanoparticles consistent with embodiments of the present disclosure was investigated. During purification of a biologic drug, the drug substance is subjected to solutions varying widely in pH and ionic strength. In order to apply viral surrogate nanoparticles across an entire downstream viral clearance process, viral surrogate nanoparticles should be stable in solutions ranging widely in pH and ionic strength. A colloidal stability assessment was performed on 20 nm viral surrogate nanoparticles across a few product-contact buffers to confirm this: A) 50 mM sodium phosphate, 2 M ammonium sulfate, pH 7.0, B) 50 mM sodium phosphate, pH 7.0, C) 20 mM tris, pH 8.0, and D) 20 mM tris, 300 mM sodium chloride, pH 8.0. In an exemplary embodiment, 20 nm viral surrogate nanoparticles were chosen for these studies due to their small size, because they can be more difficult to maintain in a colloidally stable state. Particle size distributions were measured via dynamic light scattering (DLS) (see FIG. 7A), and revealed that 20 nm viral surrogate nanoparticles were stable in all assessed environments except for buffer A. This buffering system was investigated further in a separate DLS salt-ramping study (see FIG. 7B), where it was found that viral surrogate nanoparticles are stable in ammonium sulfate concentrations up to 1 M.

Exemplary embodiments of the systems for quantifying viral surrogate nanoparticles in a solution were prepared. After performing the spiking studies and collecting the load and product process solutions from the purification processes of interest, the solutions are collected for quantification of viral surrogate nanoparticle. In these exemplary embodiments, a dead-end or “normal” flow filtration process was used to capture viral surrogate nanoparticle on a membrane surface. Following this, the fluorescence emission intensities of the post-filtration membranes were measured and used to directly calculate the viral surrogate nanoparticle concentration in the relevant starting solution via a standard curve. In some embodiments, the standard curve considers the volume of the solution filtered as well when calculating viral surrogate nanoparticle concentration from fluorescence emission intensity measurements.

Prior to beginning the normal flow filtration, the purification skid was blocked to minimize particle adsorption, e.g., to container walls, tubing, etc. In some embodiments, polyvinyl alcohol (PVA) was used as a blocking agent by preparing a 10 mg/mL PVA solution and incubating the skid with the PVA solution for one hour. Following this, the skid was rinsed with ultrapure water and dried completely. The first of the two process solutions were loaded into the pressure reservoir of a constant pressure filtration setup, and a fresh membrane was loaded into the membrane holder. The line from the pressure reservoir to the membrane holder was primed, and the membrane holder was pre-filled with ultrapure water before being connected to the setup. This helped prevent bubbles from getting trapped under the membrane. A transmembrane pressure difference of 15 psi was applied to the setup, and a weigh scale with the filtrate collection reservoir was zeroed prior to commencing the filtration. 50 mL of ultrapure water was filtered first to obtain clean water membrane flux data for the run. Following this, 100 mL of process solution was filtered, and then another 25 mL of ultrapure water was flushed through the system to push remaining process solution through the dead volume of the apparatus, and to rinse the membrane surface of any unbound viral surrogate nanoparticles. Following the filtration, the membrane was carefully removed from the holder and handled to prevent particle dislodgement. The membrane was transferred to aluminum foil with the active layer (and adsorbed viral surrogate nanoparticles) facing up and allowed to dry. This was repeated for all relevant process solutions, i.e., one nanoparticle-adsorbed membrane was produced for each process solution.

For the solid-phase fluorescence emission intensity measurement, dried, post-filtered membranes were mounted on clean glass microscopy slides using double sided tape around the membrane edges. Using a Spex® FluoroLog® Tau-3 fluorimeter (HORIBA Scientific), three emission scans of each membrane surface were performed with the sample holder set to a 30° angle from the detector, and the detector set to right angle mode. Emission scans were performed using a slit width of 3.00 nm. All measurements were taken from the center of each membrane. Emission spectra were acquired in the 500-650 nm wavelength range with a 480 nm excitation wavelength. Following fluorescence measurement, averaged fluorescence intensity data from all process solutions were compiled in Excel® for processing by Fluorescence Baseline Decomposition MATLAB code. Prior to direct calculation of particle concentration, a baseline decomposition of the acquired fluorescence intensity spectrum was used to obtain accurate and consistent viral surrogate nanoparticle fluorescence intensities, given the contribution of several components to the observed fluorescence emission spectrum of the system and the possibility of differing baselines between samples. An equation was developed to describe the observed emission spectrum as a linear combination of underlying contributions:

$${F}_{r a w} ( λ ) = a {F}_{m e m b r a n e} + b {F}_{N P} + c {λ}^{- 4} + d λ + e$$

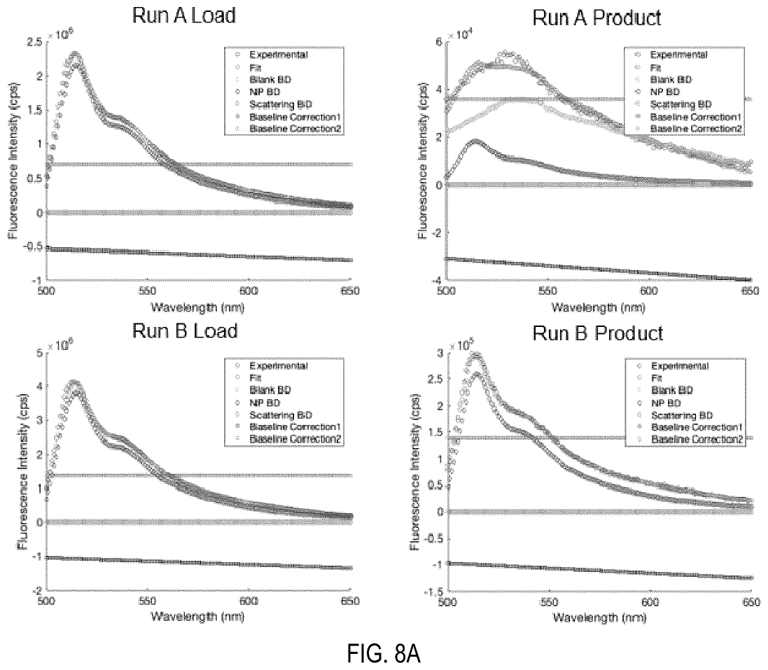

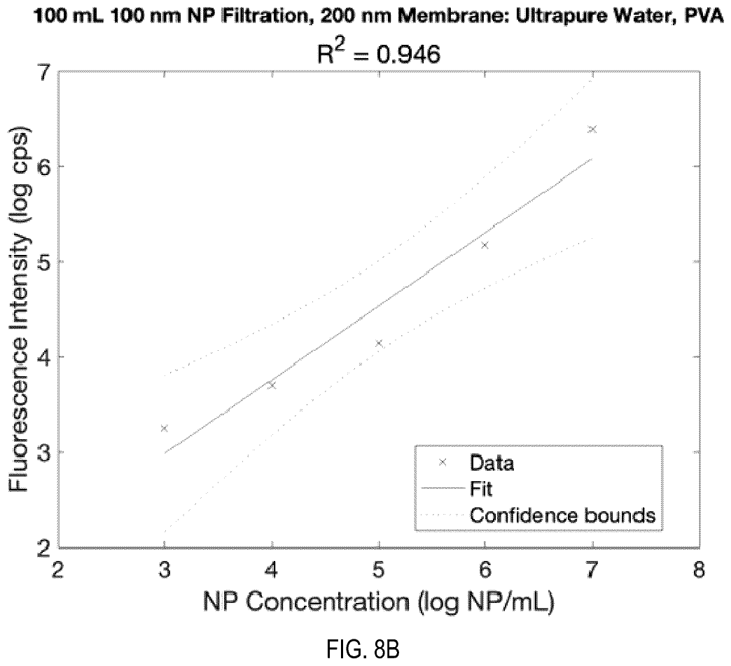

The first term accounts for the blank membrane fluorescence contributions, the second term accounts for the viral surrogate nanoparticle signal, the third term accounts for light scattering effects, and the fourth and fifth terms apply sloping baseline corrections to the data for improved sensitivity. In more detail, F_{raw} (λ) is the measured fluorescence emission intensity at a given wavelength λ, F_{membrane} (λ) and F_{NP}(λ) are the contributing fluorescence emission intensities of the bare membrane and the viral surrogate nanoparticles at wavelength λ respectively, and coefficients a through e serve as weighting terms for their respective contributing signals. This equation applies at each wavelength measured in the 500-650 nm range, forming a set of equations describing the raw, membrane and nanoparticle spectra. The raw spectra are measured for each sample; the membrane and nanoparticle spectra are measured separately for a bare membrane and a suspension of nanoparticles of know concentration, respectively. Values for the coefficients a through e are determined through a simultaneous solution of these equations via a constrained least-squares linear regression solver (e.g., lsqlin in MATLAB); the coefficients a through c are constrained to be zero or greater. The first term accounts for the blank membrane fluorescence contributions, the second term accounts for the viral surrogate nanoparticle signal, the third term accounts for light scattering effects, and the fourth and fifth terms apply sloping baseline corrections to the data to account for run-to-run variations in sample scattering and other stray light signals. The calculated coefficient b is the viral surrogate nanoparticle concentration from the decomposed viral surrogate nanoparticle signal (decoupled from all other intensity contributions). Fluorescence Baseline Decomposition Output Figures from these exemplary embodiments can be found at FIGS. 8A and 8B. Table 1 presents the final decomposed viral surrogate nanoparticle fluorescence intensities of the example data in FIG. 8A, along with the associated viral surrogate nanoparticle concentration determined through application of the standard curve (FIG. 8B).

TABLE 1
Fluorescence Baseline Decomposition
MATLAB Code Example Outputs

| Process | Baseline Decomposed | NP Solution |
|---|---|---|
| Solution ID | NP Signal (cps) | Concentration (NP/mL) |
| Run A Load | 2.16E+06 | 1.55E+07 |
| Run A Product | 1.82E+04 | 4.19E+04 |
| Run B Load | 3.80E+06 | 3.13E+07 |
| Run B Product | 2.59E+05 | 1.13E+06 |

Exemplary embodiments were prepared and investigated for dead-end flow capture based quantification of viral surrogate nanoparticles, including how pore diameter affects process parameters such as membrane porosity, permeate flux (and effectively process duration), and fouling behavior in a dead-end flow unit operation meant to capture nanoparticles. Correct membrane pore size can maximize performance of the unit operation as an analytical tool. Membrane pore sizing work was done with polycarbonate laser track-etched membranes, as polycarbonate is durable enough for handling and laser track-etching provides an isotropic, monodispersed pore structure.

Considering 20 nm viral surrogate nanoparticles, a pore size slightly smaller than that, 15 nm, was evaluated alongside 30 nm, 50 nm, and 200 nm membranes. First, scanning electron microscopy (SEM) imaging was performed to visualize the pore structure, determine the pore size distribution, and porosity for each of these membranes (Table 2). From this analysis, it was shown that porosity increased linearly with pore size. Pore density was similar across the different pore sizes evaluated, which was expected considering these membranes were manufactured using laser track-etching.

TABLE 2
Membrane Pore Size Distribution and Porosity Information

| | Measured | | |
|---|---|---|---|
| Pore Size | Diameter | # Pores/nm² | Porosity |
| (nm) | (avg nm ± 1 SD) | (avg ± 1 SD) | (A_{void}/A_{membrane}) |
| 15 | 14.97 ± 2.35 | 8.70E−06 ± 2.95E−06 | 0.002 ± 0.001 |
| 30 | 28.71 ± 4.47 | 8.81E−06 ± 1.85E−06 | 0.006 ± 0.001 |
| 50 | 51.04 ± 9.59 | 7.55E−06 ± 1.75E−06 | 0.016 ± 0.004 |
| 200 | 216.90 ± 51.53 | 2.54E−06 ± 1.27E−07 | 0.094 ± 0.009 |

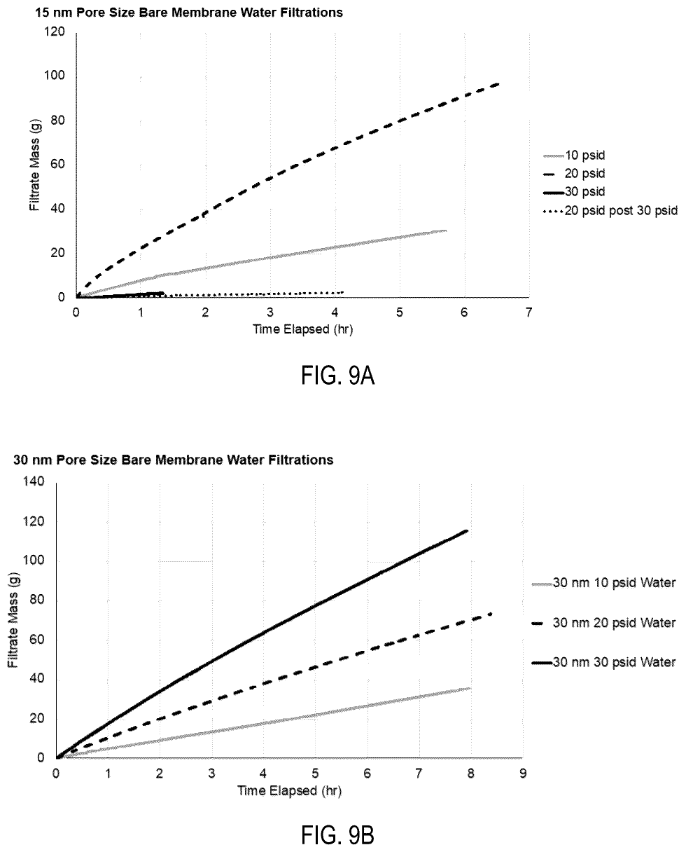

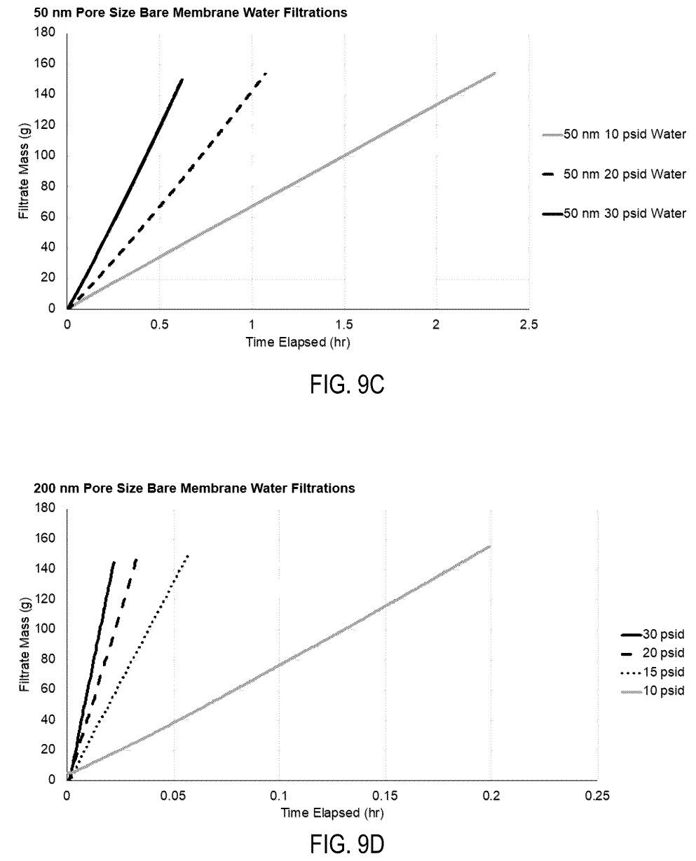

Referring now to FIG. 9A, filtrations were performed across each membrane at differential pressures ranging from 10 psid to 30 psid. Filtrations utilized ultrapure water as well as viral surrogate nanoparticles solutions in ultrapure water. For the 15 nm water filtrations, flux increased with pressure up to 20 psid. The flux decayed rapidly to 0 when operating at 30 psid; without wishing to be bound by theory, this may have been the result of membrane deformation and compaction, which likely caused complete constriction of the pores. This phenomenon showed lack of flux recovery after lowering the pressure to 20 psid across the same membrane. Membrane swelling effects could have also played a role in this. Membrane compaction and potential swelling effects were negligible for the larger membrane sizes (see FIGS. 9B-9D), where for all cases flux increased with differential pressure. Process duration significantly decreased as pore size increased. For the 15 nm and 30 nm membranes, filtering 100 mL of ultrapure water took at least 8 hours. 50 nm membranes took up to an hour and a half, 200 nm membranes took a few minutes.

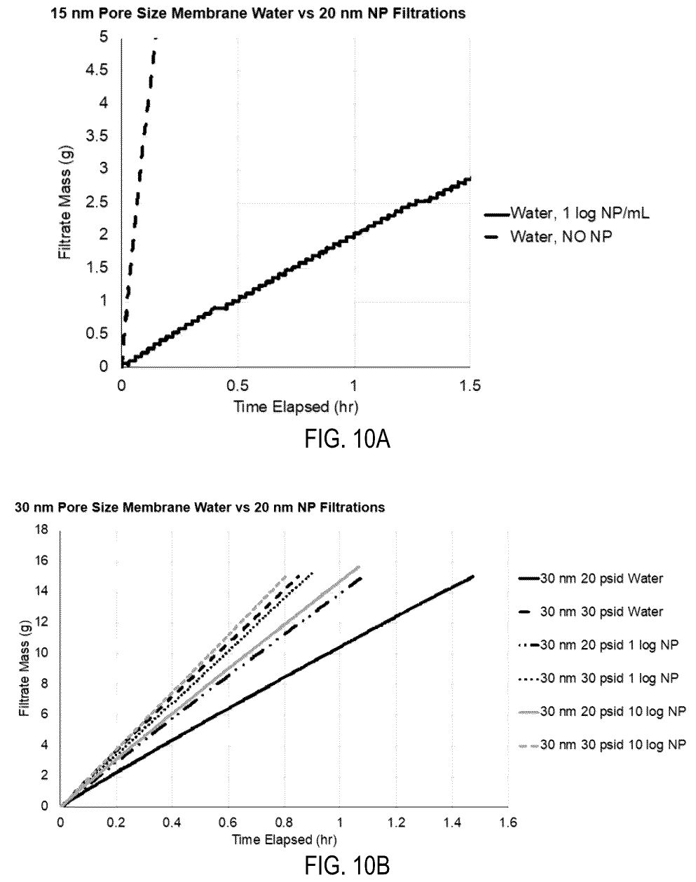

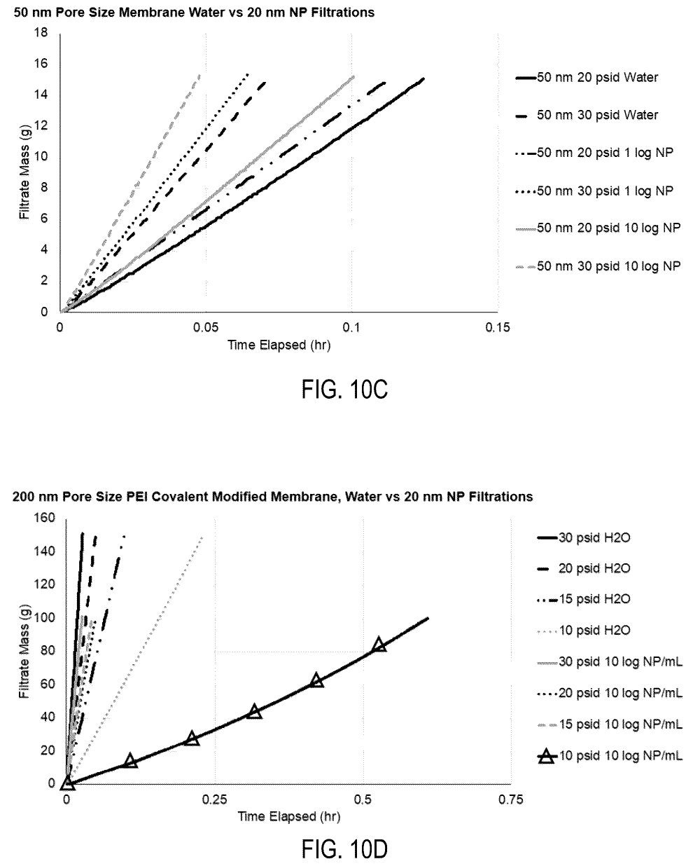

Referring now to FIG. 10A-10D, process duration for the 15 nm pore size filtration was impacted significantly by the presence of viral surrogate nanoparticles in the retentate. For the 30 nm and 50 nm pore size filtrations, the highest nanoparticle concentration filtrations had the highest flux and clean water filtrations had the lowest flux (see FIGS. 10B-10C). Without wishing to be bound by theory, this outcome was unexpected, which may be attributable to membrane-to-membrane variability. For 30 nm, 50 nm, and 200 nm filtrations, the difference in fluxes between water and viral surrogate nanoparticles runs were pressure dependent. The range of fluxes observed was higher at low pressures and decreased with increasing differential pressure. Without wishing to be bound by theory, contrasting flux behavior relative to the differential pressure across membranes of different pore sizes likely stems from the type of fouling mechanism occurring during each of these respective filtrations. For the 15 nm membranes, complete pore blockage was likely the dominating mechanism as all particles evaluated were larger than the pore diameter. In contrast, for the 200 nm pore size filtrations, either intermediate pore blockage (100+200 nm NPs) or pore constriction (20 nm NPs) followed by cake filtration fouling mechanisms were more likely as the particles were smaller than the pores. Typically for these latter mechanisms, flux decay is less severe, as many particles result in clogging a pore rather than a single particle in pore blockage. Additionally, since the systems and methods of the present disclosure adsorb particles to the membrane surface, attractive interactions between the particle and membrane surface may also play a role in the fouling behavior, leading to “random sequential adsorption” like behavior at the membrane surface.

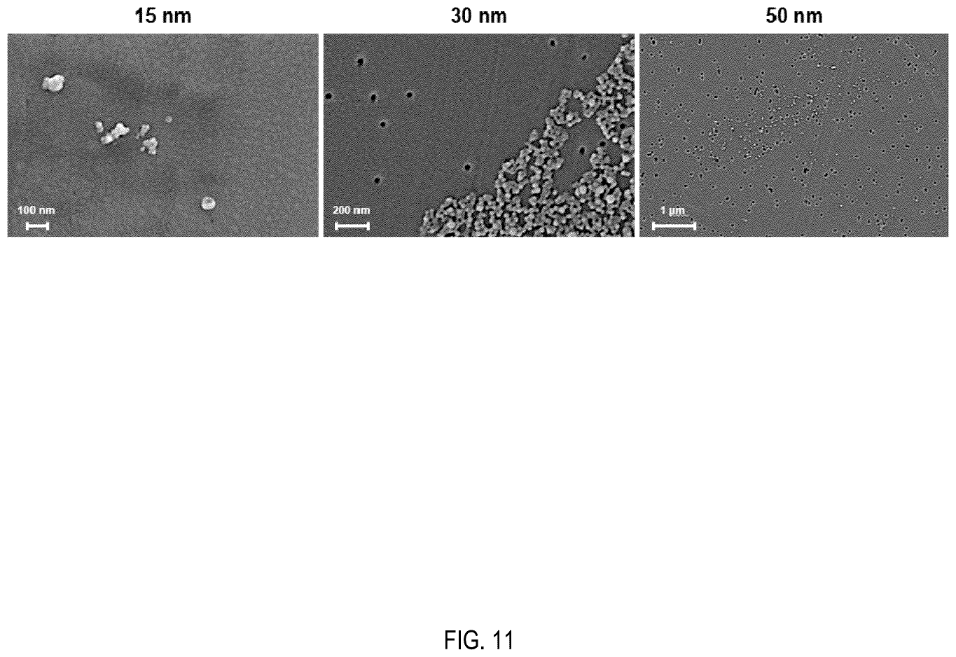

Referring now to FIG. 11, SEM images were taken of the membrane surfaces following particle filtrations in ultrapure water suspensions that showed inconsistent adsorption. Particles were successfully captured in all cases, where capture efficiencies clearly increased with decreasing pore size after taking filtrate volumes into account. However, particle adsorption was not consistent on the membrane surface. Without wishing to be bound by theory, this may have resulted from the polycarbonate membrane having the same net negative charge as the viral surrogate nanoparticles. As discussed above, in some embodiments, this can be mitigated by coating the membranes with a charged molecule or macromolecule to promote more consistent particle adsorption.

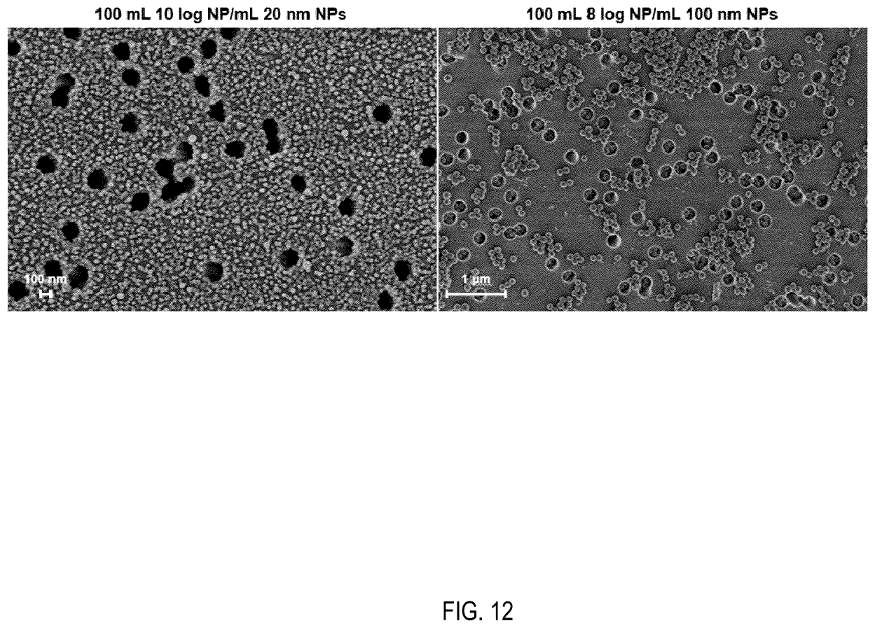

Referring now to FIG. 12, to address these inconsistencies identified above, exemplary embodiments of polycarbonate membranes functionalized with branched polyethyleneimine (PEI) for, e.g., particle quantification from dead-end flow capture, were prepared. Consistent viral surrogate nanoparticles adsorption to the membrane surface is desired for consistent nanoparticle quantification. There are multiple potential means of inducing consistent particle adsorption onto our membrane surface, with one example being the use of affinity ligands (e.g., biotin-streptavidin). In exemplary embodiments, a charge-based mechanism may be employed to adsorb viral surrogate nanoparticles. Without wishing to be bound by theory, exemplary embodiments of the viral surrogate nanoparticles of the present disclosure mimic the isoelectric points (pl) of their live virus equivalents, so naturally they are slightly negatively charged, e.g., typical virus pl is 3.5-7. Therefore, if a membrane is also negatively charged, viral surrogate nanoparticles capture efficiency is reduced. In exemplary embodiments, hydrophobic forces play a minimal role in membrane adsorption given the viral surrogate nanoparticle's net surface charge. Considering most membrane materials are also negatively charged, a protocol for coating polycarbonate track-etched membranes with a positively charged molecule was developed to help promote more consistent particle adsorption.

PEI was selected as a membrane coating in exemplary embodiments of the present disclosure. Both physical adsorption and covalent modification of the membrane surface were evaluated. Modification reactions occurred under constant agitation at 60 rpm on an orbital shaker for 24 hours at 4° C. The physical adsorption reaction utilized a reaction mixture containing the positively charged molecule diluted in ultrapure water. The covalent modification reaction was executed with a reaction mixture containing the positively charged molecule diluted in 500 mM sodium carbonate. Following the 24 hour incubation, membranes were rinsed in ultrapure water.

To visualize surface morphologies after coating, 30 nm modified membranes were imaged via SEM. Branched PEI coatings however were rough and consistent across the surface, signifying they formed somewhat more uniform coatings. Consistent particle adsorption was achieved through covalent modification of polycarbonate track-etched membrane surfaces using PEI.

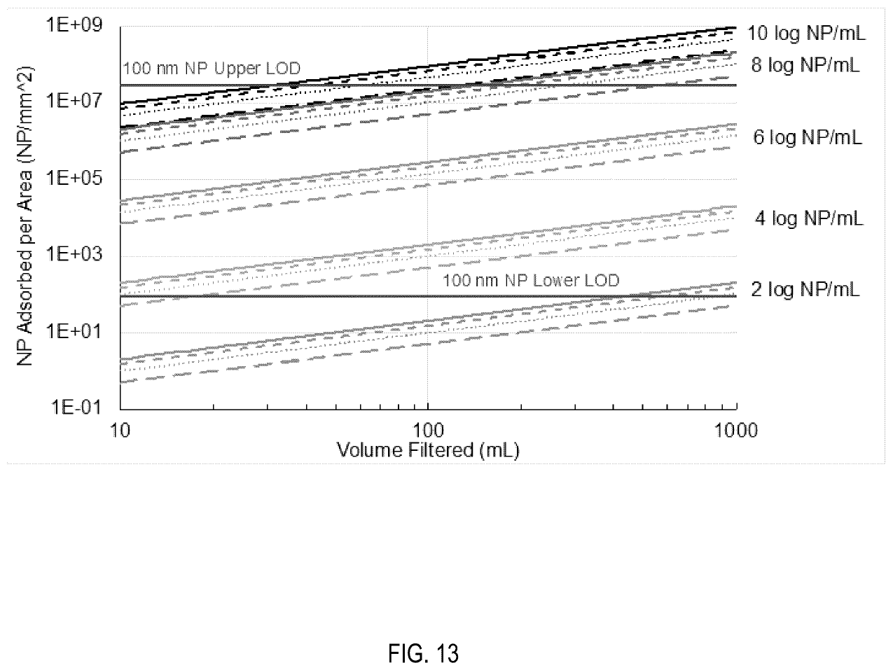

Referring now to FIG. 13, a standard curve was prepared for dead-end flow capture based quantification of viral surrogate nanoparticles in a solution consistent with exemplary embodiments of the present disclosure. Fluorescent nanoparticle concentrations can be determined via fluorescence emission spectroscopy, a baseline decomposition of the raw fluorescence signal, and the application of a standard curve. Construction of a standard curve for the dead-end flow capture assay was done by measuring the fluorescence intensity of multiple biological replicates of serially diluted sample sets of known concentrations. The resulting fluorescence intensities are then plotted with respect to nanoparticle concentration and fitted with a linear model to directly convert sample fluorescence intensities to fluorophore concentrations. When constructing the standard curve for dead-end flow capture based viral surrogate nanoparticle quantification, SEM imaging was employed along with fluorescence intensity measurements to directly relate fluorescence intensity to the number of particles visualized on the membrane surface. Therefore, a standard curve could be constructed that relates the volume filtered and fluorescence intensity to the amount of adsorbed particles, the viral surrogate nanoparticle limits of detection, and the theoretical nanoparticle solution concentration. Standard curves would be a function of both particle diameter and membrane pore size, where information contained in one bulk standard curve would apply to a particular particle diameter and pore size. To visualize, FIG. 12 portrays an approximate combined standard curve for 100 nm particles filtered across 200 nm PEI coated polycarbonate track-etched membranes.

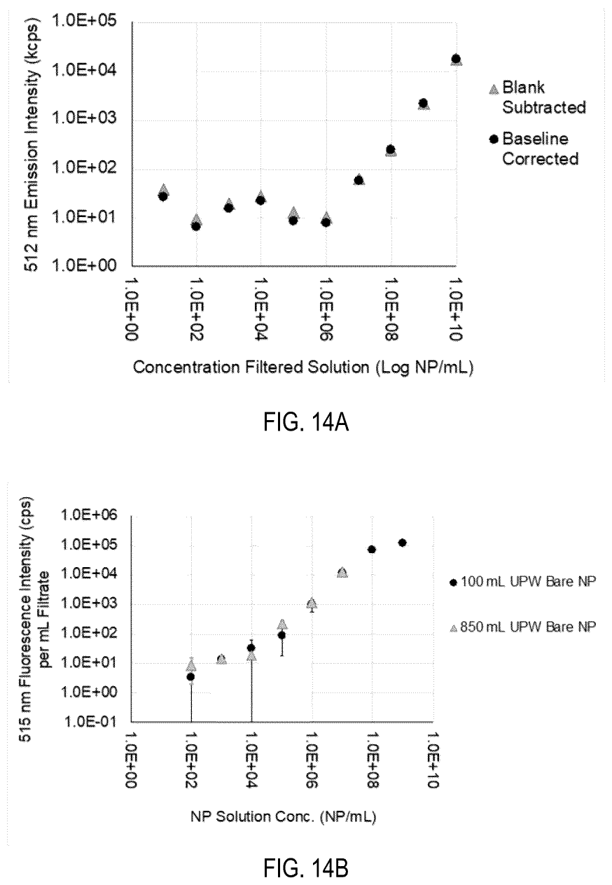

Referring now to FIGS. 14A-14B, graphs portraying fluorescence intensity versus particle concentration standard curves were generated for 100 mL filtrations with 200 nm pore size membranes for 20 nm and 100 nm nanoparticle cores. Table 3 below presents standard curve particle adsorption data obtained via SEM for the standard curves shown in FIG. 14A-14B. Here, it is shown that the limits of detection for 20 nm and 100 nm nanoparticle cores on a 200 nm membrane when processing 100 mL of solution are roughly 7 log NP/mL and 4 log NP/mL respectively. In some embodiments, these limits of detection are improved by filtering additional volume or by working with a smaller pore size membrane, as seen in FIG. 14B.

In some embodiments, blocking the surfaces of the filtration skid with polyvinyl alcohol improved the low-end limit of detection for viral surrogate nanoparticle concentration. The limit of detection of 100 nm viral surrogate nanoparticles using a 200 nm membrane was improved from 4 log NP/mL to 2 log NP/mL by employing polyvinyl alcohol (PVA) as a surface blocking agent prior to each filtration. This works to minimize particle adsorption to the walls of the skid, beneficial for consistent detection at low concentrations. When PVA is utilized, there was a tendency for it to allow for “bridging” in the larger particle systems, i.e., when one PVA polymer chain interacts with two or more particles simultaneously, generating an “apparent” aggregate and potentially having a negative impact on fluorescence. In some embodiments, the bridging effects were minimized in the presence of one or more salts.

TABLE 3
Standard Curve Particle Adsorption Density Data (SEM)

| NP | NP Sol. | Filtered | | Average | |
|---|---|---|---|---|---|
| Diameter | [ ] | Volume | | NP per | NP |
| (nm) | (log NP/mL) | (mL) | Position | Membrane | Adsorbed/mL |
| 20 | 10 | 114.2 | 1 | 1.82E+11 | 1.60E+09 |
| | | | 2 | 2.55E+11 | 2.23E+09 |
| | 9 | 114.5 | 1 | 1.50E+11 | 1.31E+09 |
| | | | 2 | 1.52E+11 | 1.33E+09 |
| | 8 | 113.4 | 1 | 2.12E+10 | 1.87E+08 |
| | | | 2 | 2.22E+10 | 1.96E+08 |
| | 7 | 114.5 | 1 | 1.49E+10 | 1.30E+08 |
| | | | 2 | 1.03E+10 | 8.97E+07 |
| | 6 | 113.8 | 1 | 6.65E+09 | 5.84E+07 |
| | | | 2 | 5.72E+09 | 5.03E+07 |
| 100 | 10 | 114 | 1 | 5.33E+10 | 4.68E+08 |
| | | | 2 | 5.29E+10 | 4.64E+08 |
| | 9 | 113.8 | 1 | 4.00E+10 | 3.52E+08 |
| | | | 2 | 4.59E+10 | 4.03E+08 |
| | 8 | 114.2 | 1 | 1.58E+10 | 1.38E+08 |
| | | | 2 | 7.62E+09 | 6.67E+07 |
| | 7 | 113.7 | 1 | 1.68E+09 | 1.47E+07 |
| | | | 2 | 1.44E+09 | 1.27E+07 |
| | 6 | 113.4 | 1 | 1.41E+08 | 1.25E+06 |
| | | | 2 | 1.72E+08 | 1.52E+06 |
| | 5 | 113.8 | 1 | 2.83E+07 | 2.48E+05 |
| | | | 2 | 2.59E+07 | 2.28E+05 |

Fluorescence emission standard curves trend with the SEM data. For the 20 nm bare particles filtered in ultrapure water, particle adsorption decreased with solution concentration until the low-end limit of detection (7 log NP/mL) was reached, where at this point particles were no longer visible on the membrane surface in images. For the 100 nm bare particles filtered in ultrapure water, high concentration nanoparticle solutions (i.e., 9 log NP/mL) saw reduced membrane fluorescence emission signal likely due to fluorophore quenching or the inner filter effect on the membrane surface. The possibility of quenching or the inner filter effect was confirmed by visualizing the membrane surface through SEM, where the membrane surface was covered by multiple layers of particles, forming a cake. SEM images of membranes that filtered mid-range 100 nm nanoparticle concentrations confirmed a linear dependence of the fluorescence intensity on adsorbed particle density. Lastly, as in the case of the 20 nm filtration, SEM images of low concentration samples were devoid of particles on the surface. Without wishing to be bound by theory, these data suggest that the linear range of quantification for this method is defined by transitions in the dominant membrane fouling mechanism over the course of the filtration. For example, a potential shift from pore constriction to intermediate pore blocking mechanisms could define the lower limit of detection for the 20 nm standard curve, and a shift from intermediate pore blocking to cake filtration could explain the departure from linearity at the upper end of the 100 nm standard curve.

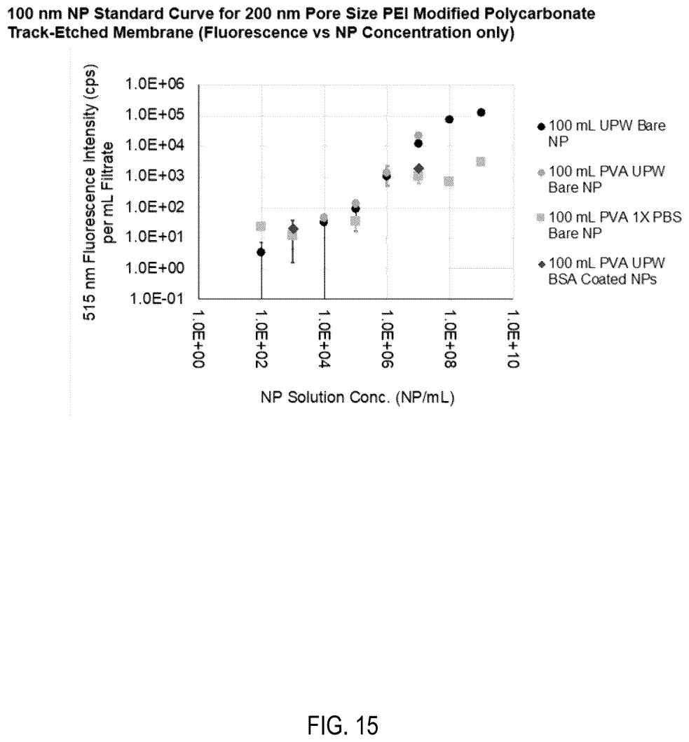

Referring now to FIG. 15, additional exemplary embodiments were prepared to investigate process solutions of varying pH, ionic strength, and general solution complexity as well as with viral surrogate nanoparticles. FIG. 15 contains standard curves for bare particles filtered in phosphate buffered saline (PBS) as well as viral surrogate nanoparticles filtered in ultrapure water. High ionic strength and surface functionalization with protein surface coverage on particles affected the high-end limit of detection in the filtration-based assay. Without wishing to be bound by theory, this may be due to charge screening in these systems, where a reduced net charge resulted in less particles sticking to the surface at higher concentrations because of a reduced long-range affinity for the surface, making multilayer formation less favorable.

Methods and systems of the present disclosure are advantageous to provide an alternative to existing viral clearance technologies. The viral surrogate nanoparticles consistent with embodiments of the present disclosure can be constructed using a polymeric base particle, where other technologies have structures more similar to virus like particles. By utilizing a polymeric base particle, better control over particle size and stability is realized. The non-enveloped embodiment can employ conjugation chemistry to covalently attach either capsid or capsid-like proteins to a fluorescent nanoparticle core. The enveloped embodiment can use conjugation chemistry to covalently attach lipid anchors to a fluorescent nanoparticle core, which allows self-assembly of glycoprotein-functionalized liposomes around the fluorescent nanoparticle for accurate mimicry of enveloped model viruses. This produces highly stable viral surrogate nanoparticles that allow for quick, simple, and precise quantification via a variety of techniques. The tunability of the viral surrogate nanoparticle surface also allows for facile expansion of viral surrogate nanoparticle technology to mimic desired virus species. The systems of the present disclosure have the versatility to mimic a variety of viruses, including both enveloped and non-enveloped viruses. Libraries of different relevant model viruses may be constructed in this manner and used in viral clearance studies, or more generally in biological studies concerning cellular uptake, accumulation, and interfacial adsorption kinetics of viral particles.

Additionally, quantification of other surrogate technologies currently involves highly cumbersome techniques with much lower sensitivity than what represents live virus clearance studies. The viral surrogate nanoparticles discussed herein present the opportunity to readily incorporate fluorescence emission properties in the particle. The fluorescent properties and significant fluorescent emission allow for precise, sensitive, high-throughput quantification, as well as imaging capability, providing a large advantage over competing technologies with respect to quantification, where these particles could be easily quantified in a high-throughput manner, e.g., by relating fluorescence intensity to particle concentration. Quantification via fluorescence would also provide high sensitivity that would be representative of current live virus clearance studies.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A system for quantifying virus concentration of a viral clearance sample, comprising:
a process solution including a target drug of interest;
a plurality of viral surrogate nanoparticles, the particles including:
a core including one or more fluorescent materials; and
a viral surface-mimicking layer on a surface of the core, wherein the viral surface-mimicking layer physicochemically mimics an external surface of a target live virus equivalent, wherein the viral surface-mimicking layer includes:
one or more capsid or capsid-like proteins of the target live virus equivalent directly associated with:
the core;
a lipid bilayer attached to the core,
or combinations thereof;
a membrane separator in fluid communication with the process solution, the membrane separator including:
a polycarbonate substrate having a plurality of pores extending therethrough; and
one or more surfaces on the substrate, the surfaces being configured to bind the viral surface-mimicking layer of the viral surrogate nanoparticles.

2. The system according to claim 1, wherein the target drug of interest includes an antibody, non-antibody protein, viral vector, nucleic acid product, blood or plasma derivative, or combinations thereof.

3. The system according to claim 1, wherein the core includes one or more polymers including polystyrene, polystyrene copolymers, or combinations thereof.

4. The system according to claim 1, wherein the viral surrogate nanoparticles mimic the size, shape, net charge, hydrophobicity, or combinations thereof, of the target live virus equivalent.

5. The system according to claim 4, wherein the cores include one or more additional chemical treatments to the surface of the core after primary surface modification, the additional chemical treatments configured further improve physicochemical mimicking of the target live virus equivalent by the viral surrogate nanoparticles.

6. The system according to claim 1, wherein the one or more capsid or capsid-like proteins are from a family including Parvoviridae, Reoviridae, Retroviridae, Herpesviridae, or combinations thereof.

7. The system according to claim 1, wherein a lipid bilayer containing embedded viral envelope proteins is attached to the core via one or more lipid anchors.

8. The system according to claim 1, wherein the core has a diameter between about 20 nm and about 200 nm.

9. The system according to claim 1, wherein the pore size of the membrane separator is 50 nm or about 200 nm.

10. The system according to claim 1, wherein the one or more surfaces on the substrate:
bears a net charge opposite of the viral surrogate nanoparticles; or
includes one or more moieties that:
promote binding of the viral surrogate nanoparticles to the membrane separator;
bear a net charge opposite of the viral surrogate nanoparticles;
or combinations thereof.

11. The system according to claim 1, further comprising a fluorescence emission spectrophotometer positioned to identify a fluorescent signal from viral surrogate nanoparticles on the membrane separator for conversion to a viral surrogate nanoparticle concentration value via a standard curve.

12. The system according to claim 1, wherein said process solution is produced from a cell culture process, a fermentation process, or combinations thereof.

13. The system according to claim 1, wherein the one or more capsid or capsid-like proteins cover about 55% of the surface of the core.

14. A method for quantifying concentration of viral particles in a process solution following viral clearance evaluation, comprising:
preparing a process solution including a target drug of interest and a plurality of viral surrogate nanoparticles configured to mimic the size, shape, net charge, hydrophobicity, or combinations thereof, of a target live virus equivalent, the particles including:
a core including one or more fluorescent materials; and
a viral surface-mimicking layer on a surface of the core, wherein the viral surface-mimicking layer physicochemically mimics an external surface of a target live virus equivalent, wherein the viral surface-mimicking layer includes:
one or more capsid or capsid-like proteins of the target live virus equivalent directly associated with:
the core;
a mock viral envelope containing envelope proteins and a
lipid bilayer,
or combinations thereof;
treating at least a portion of the process solution with one or more purification processes to form a product process solution, the one or more purification processes configured to remove one or more impurities including target live virus particles;
for quantification of viral surrogate nanoparticles in solution, contacting the product process solution with a membrane separator, the membrane separator including:
a polycarbonate substrate having a plurality of pores extending therethrough; and
one or more surfaces on the substrate, the surfaces being configured to bind the viral surface-mimicking layer of the viral surrogate nanoparticles;
and
performing a solid-phase fluorescence intensity measurement of the particles captured on the membrane separator from the product process solution to quantify the concentration of viral surrogate nanoparticles in the product process solution.

15. The method according to claim 14, further comprising:
retaining at least a portion of the process solution as a load process solution;
contacting the load process solution with a separate membrane separator;
performing a solid-phase fluorescence intensity measurement of the particles captured on each membrane separator to quantify the concentration of viral surrogate nanoparticles in each process solution; and
determining a log reduction value (LRV) of the one or more purification processes by subtracting the common log value of the concentration of viral surrogate nanoparticles in the product process solution from the common log value of the concentration of viral surrogate nanoparticles in the load process solution.

16. The method according to claim 14, wherein the target live virus belongs to a family including Parvoviridae, Reoviridae, Retroviridae, Herpesviridae, or combinations thereof.

17. The method according to claim 14, wherein the target drug of interest includes an antibody, non-antibody protein, viral vector, nucleic acid product, blood or plasma derivative, or combinations thereof.

18. A method for evaluating viral clearance of a sample, comprising:
modifying a plurality of nanoparticle cores that include one or more fluorescent materials to include a viral surface-mimicking layer, wherein the viral surface-mimicking layer physicochemically mimics the external surface of a target live virus equivalent, wherein the viral surface-mimicking layer includes:
one or more capsid or capsid-like proteins of the target live virus equivalent directly associated with:
the core;
a lipid bilayer attached to the core via one or more lipid anchors,
or combinations thereof;
modifying at least a first membrane and a second membrane in a dead-end flow nanofiltration membrane separator with one or more surfaces configured to bind the modified nanoparticle cores, the membrane including:
a polycarbonate substrate having a plurality of pores extending therethrough;
wherein at least one of the surfaces include polyethyleneimine (PEI); and
wherein the one or more surfaces are on the substrate and:
bears a net charge opposite of the modified nanoparticle cores; or
includes one or more moieties that:
promote binding of the modified nanoparticle cores to the membrane separator;
bear a net charge opposite of the modified nanoparticle cores;
or combinations thereof,
obtaining a sample from a cell culture process, a fermentation process, or combinations thereof, the sample including a target drug of interest;
performing a viral spiking study on the sample, including:
administering a concentration of the modified nanoparticle cores to the sample to form a process solution;
retaining at least a portion of the process solution as a load process solution;
performing one or more purification processes on an amount of remaining process solution to form a product process solution;
quantifying modified nanoparticle cores in the load process solution and product process solution, including:
filtering an established volume of the product process solution across the first membrane;
filtering an established volume of the load process solution across the second membrane;
performing a fluorescence intensity measurement of the modified nanoparticle cores captured on the first and second membranes;
performing a baseline decomposition of the fluorescence intensity measurements;
applying a standard curve to the baseline decomposed fluorescence data to calculate particle solution concentration; and
calculating an LRV by subtracting the common log value of the concentration of modified nanoparticle cores in the product process solution from the common log value of the concentration of modified nanoparticle cores in the load process solution,
wherein the target live virus belongs to a family including Parvoviridae, Reoviridae, Retroviridae, Herpesviridae, or combinations thereof,
wherein the target drug of interest includes an antibody, non-antibody protein, vaccine, nucleic acid product, blood or plasma derivative, or combinations thereof.

19. The method according to claim 18, wherein modifying the plurality of nanoparticle cores includes:
covalently binding the one or more capsid or capsid-like proteins using 1-ethyl-3-(3-dimethylaminopropyl)carbodiimide-N-hydroxysuccinimide conjugation chemistry to the nanoparticle cores.

20. The method according to claim 18, wherein modifying the plurality of nanoparticle cores includes:
binding primary amine-modified head group lipid anchors to the nanoparticle cores using 1-ethyl-3-(3-dimethylaminopropyl)carbodiimide-N-hydroxysuccinimide conjugation chemistry;
extruding liposomes including a lipid composition from the target live virus equivalent and membrane proteins including the one or more capsid or capsid-like proteins; and
incubating the lipid anchor-functionalized nanoparticle cores with the extruded liposomes, resulting in liposome self-assembly into a bilayer on the cores.
